(19) United States
(12) Patent Application Publication
(10) Pub. No.: US 2003/0004484 A1
(43) Pub. Date: Jan. 2, 2003

(54) Disposable absorbent article having releasable and reattachable positioning flaps

(75) Inventors: John Lee Hammons, Hamilton, OH (US); Robb Eric Olsen, Cincinnati, OH (US); Jerry Edward Carstens, West Chester, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/225,260

(22) Filed: Aug. 21, 2002

Related U.S. Application Data
(63) Continuation of application No. 09/537,847, filed on Mar. 29, 2000.

(51) Int. Cl. A61F 13/15

(52) U.S. Cl. 604/385.04; 604/381

(57) ABSTRACT

An absorbent article for positioning in the crotch area of a garment of a wearer and against or closely adjacent to the body of the wearer for absorbing body fluids. The absorbent article includes a pair of outwardly extending side flaps that are adapted to be folded around the edges of the crotch panel of the garment, and to overlap each other for attachment together to aid in properly positioning and retaining the absorbent article on the wearer's garment. Attachment of the flaps is effected by a fastening system that permits attachment, release, and subsequent reattachment of the flaps to enable the absorbent article to be repositioned by the wearer. The fastening system also enables the article to be folded for convenient carrying and packaging before use, and to be refolded for convenient disposal of the article after use. Optionally, a line of weakness can be provided across at least one of the flaps to facilitate severance of the encircling flap assembly for ease of removal of the article from a panty after use, and extensibility zones can be formed in the flaps to permit lateral extension of the flaps to accommodate a wider crotch panel of the garment and also to accommodate stresses imposed on the flaps during application of the article to the garment and while it is in use.

### FIELD OF THE INVENTION

The present invention relates to a disposable absorbent article, such as a sanitary napkin, that is intended to be worn by a user to absorb body waste fluids and that is adapted to be retained in a predetermined position on a garment that is worn by the user. More particularly, the present invention relates to a disposable absorbent article that includes side flaps that can pass around the crotch area of a garment to be releasably attached to each other and that can be detached and subsequently reattached to permit reorientation of the article relative to the garment.

### BACKGROUND OF THE INVENTION

Disposable absorbent articles in the form of sanitary napkins having laterally outwardly extending side flaps for preventing absorbed body fluids from soiling the crotch area of an undergarment are well known.

In that regard, U.S. Pat. No. 4,687,478, entitled, “Shaped Sanitary Napkin With Flaps.” which issued on Aug. 18, 1987, to Van Tilburg, shows a sanitary napkin having side flaps that form a seal with the wearer's legs to protect the wearer's undergarment from being soiled by body fluids that are not absorbed by the napkin. The flaps each have two axes of flexibility, and each flap includes an outer adhesive area for connecting the flap with the outer surface of the wearer's undergarment at the crotch area.

U.S. Pat. No. 4,589,876, entitled, “Sanitary Napkin,” which issued on May 20, 1986, to Van Tilburg, is directed to a sanitary napkin having side flaps for protecting the wearer's body and garments from being soiled by body fluids that are not absorbed by the napkin. The flaps include adhesive elements and also absorbent core members, and they are adapted to fold about a flexible fold axis to engage the outer surface of the wearer's undergarment at the crotch area.

U.S. Pat. No. 4,608,047, entitled, “Sanitary Napkin Attachment Means,” which issued on Aug. 26, 1986, to Mattingly, discloses a sanitary napkin that includes outwardly extending side flaps, one or both of which include an adhesive element for connecting the flaps together. The connection between the flaps is effected after the napkin has been positioned in the interior of the crotch area of an undergarment, by passing the flaps around the edges of the crotch area of the undergarment to meet on the outwardly-facing surface of the crotch area. Mattingly also discloses a folded sanitary napkin that utilizes the flaps to hold the folded napkin in a closed condition until it is desired to be used, and for refolding the napkin after use for disposal purposes.

Although the prior art napkins are generally suitable for performing their intended functions, it is desirable to provide an improved sanitary napkin that can be conveniently folded and packaged before use, that enables the napkin to be securely repositioned when in use and after it has at least once been positioned, and that is refoldable for convenient disposal after use.

It is therefore an object of the present invention to provide an improved disposable absorbent article having laterally outwardly extending side flaps that are of a size sufficient to permit them to pass around the outside of the crotch area of an undergarment for attachment to each other, and that can thereafter be detached from each other and subsequently reattached again to remain reattached.

It is another object of the present invention to provide a disposable absorbent article having laterally extending side flaps that can be folded over the absorbent article before use to protect the absorbent material carried by the article, and that also can be folded over the absorbent material and attached to each other after use of the article, to enable convenient and sanitary disposal of the used absorbent article.

It is a further object of the present invention to provide a disposable absorbent article having laterally outwardly extending side flaps that include a line of weakness to enable rapid and easy severance of the connected flaps after use of the article, to facilitate removal of the used article from a garment after use.

It is a still further object of the present invention to provide a disposable absorbent article having laterally outwardly extending side flaps, wherein particular areas of the side flaps are extensible to enable the flaps to extend as necessary to adapt to specific use-influenced stress conditions imposed on the flaps.

### SUMMARY OF THE INVENTION

Briefly stated, in accordance with one aspect of the present invention, a disposable absorbent article is provided. The article is adapted to be worn and to be positioned relative to the wearer's body for receiving and retaining body waste fluids to prevent soiling of undergarments worn by the wearer. The absorbent article includes a liquid-impervious backsheet having a rear surface adapted to face toward the wearer's garments when the absorbent article is worn, and a front surface adapted to face toward the wearer's body when the absorbent article is worn, the backsheet including a pair of longitudinally extending side edges. An elongated absorbent core is provided for absorbing body waste fluids of the wearer while the absorbent article is worn, and is positioned in overlying relationship with the front surface of the backsheet. A liquid-pervious topsheet overlies the absorbent core and has a rear surface facing toward the absorbent core and a front surface facing toward and adapted to contact the wearer's body when the article is worn. A pair of side flaps extend laterally outwardly from the side edges of the absorbent core, each flap having a liquid-impervious outer surface adapted to face toward the wearer's garments when the absorbent article is worn, and an inner surface adapted to face toward the wearer's body when the absorbent article is worn, wherein each of the flaps extends laterally from the absorbent core longitudinal axis a distance sufficient to enable the flaps to be in at least partial overlapping relationship when each of the flaps is folded around respective edges of a crotch area of the undergarment after initial placement of the backsheet of the absorbent article in a desired position against the inner crotch surface of the undergarment, with at least one side flap having an area of elastic extensibility defined by a plurality of laterally spaced, longitudinally extending, parallel ridges and alternating parallel grooves formed in the at least one side flap to allow the at least one side flap to elastically extend in response to stresses applied to the at least one side flap when the absorbent article is placed in position against the inner crotch surface of the undergarment.

In accordance with another aspect of the present invention a line of weakness is formed in at least one side flap of the absorbent article to facilitate rapid and trouble-free removal of the article after use.

In accordance with a further aspect of the present invention, a method of folding a disposable absorbent article is provided to enclose the body-contacting surfaces thereof both before and after use to maintain cleanliness.

### BRIEF DESCRIPTION OF THE DRAWINGS

FIG. 1 is a top plan view of a sanitary napkin in accordance with the present invention, the napkin including side flaps having an adhesive area on one flap and a contact area on the other flap.

FIG. 2 is an enlarged, cross-sectional view of the sanitary napkin shown in FIG. 1, taken along the line 2-2 thereof.

FIG. 3 is a fragmentary, top plan view showing an alternative configuration for the side flaps.

FIG. 4 is a fragmentary view similar to that of FIG. 3 and showing another configuration for the side flaps.

FIG. 5 is a fragmentary perspective view of the crotch area of a panty within which a sanitary napkin in accordance with the present invention can be positioned for use.

FIG. 6 is a view similar to FIG. 4 showing a sanitary napkin in position within the panty.

FIG. 7 is a top plan view of another embodiment of a sanitary napkin in accordance with the present invention, the napkin including side flaps and having both an adhesive area and a contact area on each flap.

FIG. 8 is a cross-sectional view of the sanitary napkin shown in FIG. 7, taken along the line 8-8 thereof.

FIG. 9 is a top plan view of a further embodiment of a sanitary napkin in accordance with the present invention, the napkin including side flaps and having both an adhesive area and a contact area on each flap, wherein the adhesive areas and contact areas are oriented differently relative to each other.

FIG. 10 is a cross-sectional view of the sanitary napkin shown in FIG. 9, taken along the line 10-10 thereof.

FIG. 11 is a top plan view of still another embodiment of a sanitary napkin in accordance with the present invention, the napkin including side flaps and having both an adhesive area and a contact area on each flap, wherein the adhesive areas and the contact areas are each rotated 90° relative to their positions as shown in the FIG. 9 embodiment.

FIG. 12 is a cross-sectional view of the sanitary napkin shown in FIG. 11, taken along the line 12-12 thereof.

FIG. 13 is a top plan view of another embodiment of a sanitary napkin in accordance with the present invention, the napkin including side flaps defined by a separate crosspiece attached to the napkin.

FIG. 14 is a cross-sectional view of the sanitary napkin shown in FIG. 13, taken along the line 14-14 thereof.

FIG. 15 is a top plan view of still another embodiment of a sanitary napkin in accordance with the present invention, the napkin including side flaps each defined by individual crosspieces attached to the napkin.

FIG. 16 is a cross-sectional view of the sanitary napkin shown in FIG. 15, taken along the line 16-16 thereof.

FIG. 17 is a top plan view of another embodiment of a sanitary napkin in accordance with the present invention, the napkin including side flaps each having one form of localized extensible areas.

FIG. 18 is a cross-sectional view of the sanitary napkin shown in FIG. 17, taken along the line 18-18 thereof.

FIG. 19 is a top plan view of a further embodiment of a sanitary napkin in accordance with the present invention, the napkin including side flaps each having another form of localized extensible areas.

FIG. 20 is a cross-sectional view of the sanitary napkin shown in FIG. 19, taken along the line 20-20 thereof.

FIG. 21 is a top plan view of another embodiment of a sanitary napkin in accordance with the present invention, the napkin including side flaps each having still another form of localized extensible areas.

FIG. 22 is a cross-sectional view of the sanitary napkin shown in FIG. 21, taken along the line 22-22 thereof.

FIG. 23 is a top plan view of a further embodiment of a sanitary napkin in accordance with the present invention, the napkin including side flaps each having a further form of localized extensible areas.

FIG. 24 is a cross-sectional view of the sanitary napkin shown in FIG. 23, taken along the line 24-24 thereof.

FIGS. 25 through 28 are top plan views of a sanitary napkin in accordance with the present invention at various stages of a folding operation to fold the napkin into a self-contained package.

FIG. 29 is a top plan view of another form of sanitary napkin in accordance with the present invention.

FIG. 30 is a top plan view of the sanitary napkin shown in FIG. 29 after two steps of a folding operation to form the napkin into a convenient, self-contained package.

FIG. 31 is a right side view of the sanitary napkin shown in FIG. 30.

FIG. 32 is a top plan view of the napkin shown in FIGS. 29 through 31 after is has been folded into a self-contained package.

FIG. 33 is a top plan view of another form of sanitary napkin in accordance with the present invention.

FIG. 34 is a top plan view of the sanitary napkin shown in FIG. 33 after three steps of a folding operation to form the napkin of FIG. 33 into a convenient, self-contained package.

FIG. 35 is a top plan view similar to FIG. 34 after a fourth fold of a folding operation.

FIG. 36 is a top plan view similar to FIG. 35 after a fifth fold of a folding operation.

FIG. 37 is a top plan view of the napkin shown in FIGS. 33 through 36 after is has been folded into a self-contained package.

FIG. 38 is a top perspective view of another form of sanitary napkin in accordance with the present invention.

FIG. 39 is a top plan view of the napkin of FIG. 38 after the ends have been folded inwardly.

FIG. 40 is a top perspective view of the napkin of FIG. 38 after the flaps have been folded over the infolded ends.

FIG. 41 is a view toward the crotch area of an undergarment with the flaps of a sanitary napkin in accordance with the present invention attached to each other and showing a line of weakness to facilitate separation of the flaps for removal of the napkin from the undergarment.

FIG. 42 is a view similar to that of FIG. 41, but showing a further embodiment of the present invention including a separate attachment piece spanning a pair of folded-over flaps.

### DETAILED DESCRIPTION OF THE INVENTION

As used herein, the following terms have the following meanings:

“absorbent article” applies to an article that is intended to be worn by a person or to be carried on the body of a person in such a position on the body as to absorb and to contain bodily exudates and discharges;

“disposable” applies to an absorbent article that is not intended to be laundered or otherwise treated or restored to enable reuse as an absorbent article, but that is intended to be discarded after a single use, and, preferably, to be recycled, composted, or otherwise disposed of in an environmentally acceptable manner;

“unitary” as applied to an absorbent article refers to an absorbent article that is formed of separate parts that are joined or united together in such a way as to form a coordinated entity so that it does not require adjuncts or separate and distinct parts, such as a separate holder and pad, in order to function for the intended purpose;

“sanitary napkin” refers to a unitary, disposable, absorbent article that is worn by females at the pudendal region adjacent to the externally visible female genitalia, generally external to the urogenital region, and that is intended to absorb and to contain fluids, such as menstrual fluids, urine, and other vaginal discharges that emanate from the wearer's body; it includes other feminine hygiene or catamenial pads, such as articles sometimes referred to as “pantiliners,” other absorbent articles, such as absorbent pads sometimes referred to as incontinence pads, interlabial devices that are positioned partially within and partially without the wearer's vaginal cleft, and the like.

### Overall Structure of the Absorbent Article

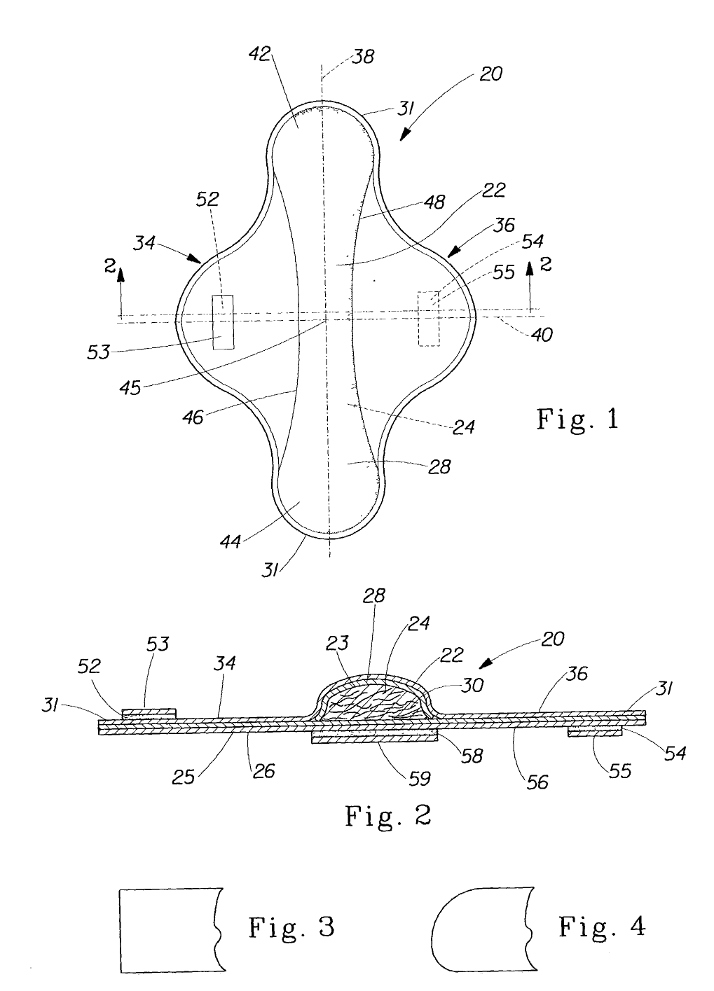

A preferred embodiment of a disposable absorbent article in accordance with the present invention is a catamenial pad or sanitary napkin that can have a structure of the type shown in the several drawing figures. Referring particularly to FIGS. 1 and 2 thereof, there is shown a unitary, disposable, absorbent article 20 in accordance with the present invention. Article 20 includes a flat, thin, flexible, liquid-permeable topsheet 22, and a fluid-absorbent core 24 that is thicker than topsheet 22, a flat, thin, liquid-impervious backsheet 26. As seen in FIG. 2, the major outwardly-facing surfaces of article 20 are defined by topsheet 22 and by backsheet 26. Absorbent core 24 is positioned between topsheet 22 and backsheet 26.

When absorbent article 20 is in use, topsheet 22 is intended to be positioned against or closely adjacent to the body of a wearer at or closely adjacent to a point where liquid is discharged from the body, so that the discharged liquid can pass through topsheet 22 and can be absorbed by and collected in absorbent core 24. Backsheet 26 is outermost, relative to the body of the wearer, and it serves to prevent liquid absorbed by and carried by absorbent core 24 from wetting a garment that is worn by the wearer adjacent to absorbent article 20.

FIG. 1 is a top plan view of absorbent article 20 in its flat, opened condition, before it is positioned for use adjacent the body of the wearer. Topsheet 22 is uppermost, relative to the viewer, and backsheet 26 is lowermost. Thus, in the orientation of absorbent article 20 as shown in FIG. 1, the uppermost, liquid-absorbing surface 28 of absorbent core 24, the surface that is in contact with topsheet 22 and that faces and is adjacent the wearer's body when article 20 is in use, is shown as facing the viewer of FIG. 1. Conversely, the lowermost surface 30 of absorbent core 24, that faces away from the body of the wearer when absorbent article 20 is in use, is in contact with backsheet 26, and therefore lowermost surface 30 is not visible in FIG. 1.

FIG. 2 is a cross-sectional view that more clearly shows the overall structure of absorbent article 20 and the relative positions of topsheet 22, absorbent core 24, and backsheet 26.

### Backsheet Structure

Backsheet 26 is impervious to liquids (e.g., menses and/or urine) and prevents the liquids that are absorbed by and contained in absorbent core 24 from wetting clothing articles such as pants, pajamas, and undergarments, clothing articles that could come into contact with absorbent article 20. Preferably, backsheet 26 is made from a thin, flexible, liquid-impervious plastic film, although other flexible, liquid-impervious materials can also be employed.

As used herein, the term “flexible” refers to materials that are compliant and will readily conform to the general shape and contours of the human body. Backsheet 26 can thus be made from a soft, flexible, liquid-impervious woven or nonwoven material; from a flexible, liquid-impervious polymeric film, for example a thermoplastic film such as polyethylene or polypropylene; or from a soft, flexible, liquid-impervious composite material, such as a film-coated nonwoven material.

Preferably, backsheet 26 is formed from a thin, polyethylene film having a thickness of from about 0.012 mm (0.5 mil) to about 0.051 mm (2.0 mils). Examples of suitable polyethylene films are those films manufactured by Clopay Corporation, of Cincinnati, Ohio, and sold under the designation P18-0401, and by Tredegar Industries, Inc., of Terre Haute, Ind., and sold under the designation XP-39385. If provided in the form of a polymeric film, backsheet 26 can advantageously be embossed with a predetermined embossing pattern, or it can be matte finished, to provide a more cloth-like appearance to its surface. Additionally, backsheet 26 can optionally be made from a flexible, liquid-impervious material that permits vapors to pass therethrough. Therefore, when such a backsheet is employed, vapors can escape from absorbent core 24 and can pass through backsheet 26, because the backsheet is breathable, while at the same time preventing liquids and other exudates from passing from absorbent core 24 through the backsheet.

### Absorbent Core Structure

Absorbent core 24 can be any material that is capable of absorbing and retaining bodily liquids (e.g., menses and/or urine). For example, absorbent core 24 can be made from a wide variety of liquid-absorbent materials of the type commonly found in sanitary napkins and in other disposable absorbent articles. Specific examples of suitable absorbent materials include comminuted wood pulp, which is generally referred to as “airfelt;” creped cellulose wadding; meltblown polymers, including coform; chemically stiffened, modified, or cross-linked cellulosic fibers; synthetic fibers, such as crimped polyester fibers; peat moss; tissue, including tissue wraps and tissue laminates; absorbent foams; absorbent sponges; superabsorbent polymers; absorbent gelling materials; any equivalent material capable of absorbing liquids; and combinations or mixtures of the foregoing materials.

The configuration and construction of absorbent core 24 can also be changed from that shown in the drawings. For example, absorbent core 24 can be formed in a wide variety of sizes and shapes, including, but not limited to, rectangular, oval, hourglass, dog bone, asymmetric, and the like, and it can have zones of different thicknesses at different portions of the core, such as a profile that is thicker in the center of absorbent article 24 than adjacent its edges. Additionally, absorbent core 24 can have hydrophilic gradients, superabsorbent gradients, or lower density and lower average basis weight acquisition zones, or it can include one or more layers or additional structures. Preferably, however, the liquid-absorbing capacity of absorbent core 24 is made to be compatible with the intended loading of liquid, which is based upon the intended duration and conditions of use of the absorbent article. In that regard, the physical size and the absorbent capacity of the absorbent core can be changed to accommodate different use conditions of the absorbent article, such as incontinence pads, pantiliners, regular sanitary napkins, or overnight sanitary napkins.

Additional examples of absorbent structures that can be used to provide the absorbent core of a sanitary napkin in accordance with the present invention are described in U.S. Pat. No. 4,950,264, entitled, “Thin, Flexible Sanitary Napkin,” which issued on Aug. 21, 1990, to Osborn; U.S. Pat. No. 4,610,678, entitled, “High-Density Absorbent Structures,” which issued on Sep. 9, 1986, to Weisman et al.; U.S. Pat. No. 4,834,735, entitled, “High Density Absorbent Members Having Lower Density and Lower Basis Weight Acquisition Zones,” which issued on May 30, 1989, to Alemany et al.; and European Patent Application No. 0 198 683, which was published on Oct. 22, 1986, in the name of The Procter & Gamble Company as applicant, and identifying Duenk et al. as inventors. The disclosures of each of the foregoing patents are hereby incorporated herein by reference to the same extent as if fully rewritten.

### Topsheet Structure

Topsheet 22 is preferably compliant, soft feeling, and non-irritating to the wearer's skin, and it is necessarily liquid pervious to permit liquids (e.g., menses and/or urine) to readily penetrate through its thickness and into the absorbent core. A suitable topsheet 22 can be made from a wide range of liquid-pervious materials, including woven and nonwoven materials; polymeric materials, such as apertured, formed thermoplastic films, apertured plastic films, and hydroforned thermoplastic films; porous foams; reticulated foams; reticulated thermoplastic films; and thermoplastic scrims. Suitable woven and nonwoven materials can include natural fibers (e.g., wood or cotton fibers), synthetic fibers (e.g., polymeric fibers such as polyester, polypropylene, or polyethylene fibers), or a combination of natural and synthetic fibers.

A preferred topsheet structure is an apertured, formed, liquid-pervious film. Apertured, formed films are preferred for the topsheet because they are pervious to body exudates, yet they are non-absorbent and therefore they do not feel wet. Additionally, such films advantageously exhibit a reduced tendency to allow liquids that pass therethrough to pass back from the absorbent core through the topsheet to wet the wearer's skin. Thus, the surface of an apertured, formed film that is in contact with the body remains dry, thereby reducing body soiling and resulting in a more comfortable feel for the person wearing the absorbent article. Suitable apertured, formed films are described in U.S. Pat. No. 3,929,135, entitled, “Absorptive Structures Having Tapered Capillaries,” which issued on Dec. 30, 1975, to Thompson; in U.S. Pat. No. 4,324,246, entitled, “Disposable Absorbent Article Having A Stain Resistant Topsheet”, which issued on Apr. 13, 1982, to Mullane, et al.; in U.S. Pat. No. 4,342,314, entitled, “Resilient Plastic Web Exhibiting Fiber-Like Properties,” which issued on Aug. 3, 1982, to Radel et al.; in U.S. Pat. No. 4,463,045, entitled, “Macroscopically Expanded Three-Dimensional Plastic Web Exhibiting Non-Glossy Visible Surface and Cloth-Like Tactile Impression,” which issued on Jul. 31, 1984, to Ahr et al.; and in U.S. Pat. No. 5,006,394, entitled, “Multilayer Polymeric Film,” which issued on Apr. 9, 1991, to Baird. The disclosures of each of the foregoing patents are hereby incorporated herein by reference to the same extent as if fully rewritten.

The preferred topsheet for the present invention is an apertured, formed film as described in one or more of the foregoing patents and also those topsheets included as a part of sanitary napkins made and sold by The Procter & Gamble Company, of Cincinnati, Ohio, and known as “DRI-WEAVE.”

In a highly preferred embodiment of the present invention, the body-facing surface of the apertured, formed film topsheet is hydrophilic to facilitate liquid transfer through the topsheet at a faster rate than if the body surface were not hydrophilic. That property of the topsheet reduces the likelihood that menstrual or other fluid will flow off the surface of the topsheet, rather than flowing through it and being absorbed by the absorbent core as intended. In one preferred embodiment, a surfactant is incorporated into the polymeric material of the apertured, formed film topsheet, as is described in U.S. Statutory Invention Registration No. H1,670, entitled, “Absorbent Article Having A Nonwoven and Apertured Film Coversheet,” published on Jul. 1, 1997, in the names of Aziz, et al. as inventors, the disclosure of which is hereby incorporated herein by reference to the same extent as if fully rewritten.

Alternatively, the body-facing surface of the topsheet can be made hydrophilic by treating it with a surfactant in the manner described in U.S. Pat. No. 4,950,254, entitled, “Thin, Flexible Sanitary Napkin,” which issued on Aug. 21, 1990, to Osborn, the disclosure of which is hereby incorporated herein by reference to the same extent as if fully rewritten.

### Assembly of the Several Elements of the Structure

Topsheet 22 and backsheet 26 are preferably coextensive with each other and have respective outermost edges that are joined to each other along a joinder seam 31 that extends either outwardly of or adjacent the peripheral edges of absorbent core 24, so that absorbent core 24 is positioned and is retained between topsheet 22 and backsheet 26. Additionally, backsheet 26 and topsheet 22 are also preferably joined both to absorbent core 24, and to each other outwardly of absorbent core 24, by a suitable attachment arrangement (not shown), a number of which attachment arrangements are known to those having skill in the art. For example, backsheet 26 or topsheet 22, or both, can be secured to absorbent core 24, or to each other, by a uniformly thick, continuous layer of adhesive, a patterned, discontinuous layer of adhesive, or an array of spaced lines, spirals, spots, or other spaced forms of discrete adhesive areas. Adhesives that have been found to be satisfactory for that purpose are manufactured by the H. B. Fuller Company, of St. Paul, Minn., under the designations HL-1258 and H-2031.

A preferred attachment arrangement for joining the backsheet and the topsheet includes an open pattern network of filaments of adhesive, as is disclosed in U.S. Pat. No. 4,573,986, entitled, “Disposable Waste-Containment Garment”, which issued on Mar. 4, 1986, to Minetola, et al., the disclosure of which is hereby incorporated herein by reference to the same extent as if fully rewritten. An example of another form of suitable attachment arrangement is an open pattern network of filaments that includes several lines of adhesive filaments swirled into a spiral pattern that can be applied by the apparatus and method shown in U.S. Pat. No. 3,911,173, which issued on Oct. 7, 1975, to Sprague, Jr.; in U.S. Pat. No. 4,785,996, entitled, “Adhesive Spray Gun and Nozzle Attachment,” which issued on Nov. 22, 1978, to Ziecker, et al.; and in U.S. Pat. No. 4,842,666, entitled, “Process for the Permanent Joining of Stretchable Threadlike or Small Ribbonlike Elastic Elements to a Flat Substrate, as well as Use Thereof for Producing Frilled Sections of Film of Foil Strip,” which issued on Jun. 27, 1989, to Werenicz. The disclosures of each of the foregoing patents are hereby incorporated herein by reference to the same extent as if fully rewritten.

Alternatively, the attachments between the backsheet, the topsheet, and the absorbent core can be effected in the form of heat bonds, pressure bonds, ultrasonic bonds, dynamic mechanical bonds, or any other suitable attachment arrangements that are known to those having skill in the art, and combinations of any such attachments.

Although topsheet 22, backsheet 26, and absorbent core 24 can be formed and assembled in a variety of well-known configurations (including so called “tube” products or so-called “side flap” products), a preferred sanitary napkin structural arrangement of the several elements of such articles to provide the benefits of the present invention can conform generally with those structures described in U.S. Pat. No. 4,950,264, entitled, “Thin, Flexible Sanitary Napkin,” which issued on Aug. 21, 1990, to Osborn; U.S. Pat. No. 4,425,130, entitled, “Compound Sanitary Napkin,” which issued on Jan. 10, 1984, to DesMarais; U.S. Pat. No. 4,321,924, entitled, “Bordered Disposable Absorbent Article,” which issued on Mar. 30, 1982, to Ahr; and U.S. Pat. No. 4,687,478, entitled, “Shaped Sanitary Napkin With Flaps,” which issued on Aug. 18, 1987, to Van Tilburg. In that regard, the disclosure of each of the foregoing patents is hereby incorporated herein by reference to the same extent as if fully rewritten.

### Liquid Transport Layer

Optionally, one or more transport layers 23 (see FIG. 2) can be positioned between topsheet 22 and absorbent core 24, or it or they can be attached to the garment-facing surface of topsheet 22 to form a composite topsheet. A transport layer can perform several functions, including spacing absorbent core 24 a slightly greater distance from the skin of the wearer, to further reduce the likelihood of the wearer experiencing a feeling of wetness, and also improving the lateral transport of liquid exudates across and along upper surface 28 of absorbent core 24 by a wicking effect. Improved lateral transport or wicking of liquid exudates over the surface of absorbent core 24 is desirable because it promotes a more even distribution of the liquid exudates into the interior of absorbent core 24. As a consequence, transport layer 23 enables absorbent article 20 to be thinner than it would otherwise be, because the entire absorptive capacity of absorbent core 24 can be more efficiently utilized for liquid absorption, as opposed to absorption only within a local area of the absorbent core at the point where liquid exudates first contact the absorbent article. The wicking effect referred to herein includes the transportation of liquids to, along the surface of, and into the absorbent core in one, two, or all directions (i.e., in the x-y plane and/or in the z-direction).

Transport layer 23 can be made from several different materials, including thin, nonwoven or woven webs of synthetic fibers that can also include polyester, polypropylene, or polyethylene; natural fibers, such as cotton or cellulose; blends of natural and synthetic fibers; and any equivalent materials or combinations of materials. The structures of sanitary napkins having both a transport layer and a topsheet are more fully described in U.S. Pat. No. 4,950,264, entitled, “Thin, Flexible Sanitary Napkin,” which issued on Aug. 21, 1990, to Osborn; and in published PCT application No. PCT/US92/09716, entitled, “Absorbent Article Having Fused Layers,” published on Jun. 24, 1993, as International Publication No. WO93/11725. The disclosures of each of those publications are hereby incorporated herein by reference to the same extent as if fully rewritten. In a preferred embodiment, transport layer 23 is joined with topsheet 22 by any of the conventional means for joining webs together, most preferably by fusion bonds, as is more fully described in the above-identified PCT publication.

### Additional Absorbent Sheet

A flat, thin, absorbent lower sheet can optionally be positioned between topsheet 22 and backsheet 26, particularly in the areas defined by the outwardly-extending side flaps 34, 36. In the embodiment illustrated in FIGS. 1 and 2, which shows such a flat, thin, absorbent lower sheet 25 in position between topsheet 22 and backsheet 26. Absorbent lower sheet 25 extends over the body-facing surfaces or each of the flaps, and it also underlies absorbent core 24. That positional arrangement is preferred for convenience of manufacture. It also enhances the functioning of the absorbent article by providing additional absorption capacity. Absorbent lower sheet 25 can absorb body fluids that are not absorbed by absorbent core 24, or that are expressed from core 24 while the article is worn and as a consequence of the wearer's movements. Absorbent lower sheet 25 is generally compressible, flexible, and non-irritating to the wearer's skin. It can be made from any of the materials that are identified above for making absorbent core 24. Absorbent lower sheet 25 can also be made from a tissue paper sheet, if desired, to minimize the overall thickness of the flaps, but it can also be a thicker absorbent material, if desired.

### Description of the FIG. 1 Embodiment

Referring once again to FIG. 1, there is shown an embodiment of an absorbent article 20 in the form of a sanitary napkin in which absorbent core 24 is of elongated form and has a longitudinal central axis 38 and a transverse central axis 40. As shown, absorbent core 24 is symmetrical about each of axes 38 and 40, and it has rounded ends 42 and 44. Furthermore, as also seen in FIG. 1, absorbent core is wider at its longitudinal ends 42, 44 than it is at its center 45.

As used herein, the term “longitudinal” refers to a line, an axis, or a direction in the plane of absorbent article 20 that is generally aligned with (e.g., approximately parallel to) a vertical plane that passes through a standing wearer of the article to bisect the wearer into substantially equal left and right body halves. As used herein, the terms “transverse” and “lateral” are interchangeable with each other and refer to a line, an axis, or a direction that lies within a plane of absorbent article 20 that is generally perpendicular to the longitudinal line, axis, or direction.

Preferably, topsheet 22 and backsheet 26 each have respective length and width dimensions that are larger than the corresponding dimensions of absorbent core 24. Thus, topsheet 22 and backsheet 26 each extend beyond each of ends 42, 44 of absorbent core 24, and also beyond its side edges 46, 48, to thereby define not only outer or peripheral portions of absorbent article 20, but also a pair of laterally outwardly extending side flaps 34 and 36.

### Side Flap Configuration and Structure

Side flaps 34 and 36 are adjacent to and extend outwardly from respective side edges 46, 48 of absorbent core 24. When absorbent article 20 is placed in its use position within the crotch region of an undergarment (see FIG. 6), flaps 46, 48 each loosely drape over the inner edges of the respective leg openings of the wearer's undergarment, or panty, at the crotch region of the garment, so that the flaps are positioned between the respective inner edge portions of each of the leg openings of the wearer's panty and the inner portions of each of the wearer's thighs when the panty is worn and the absorbent article is in its preferred position adjacent the wearer's body. The lateral distance to which flaps 46 and 48 extend outwardly from the core is a distance sufficient to permit the flaps to at least partially overlap when each of the flaps is folded over the respective inner edge of a leg opening of the panty and the flaps are positioned in overlapping relationship against the outwardly-facing surface of the crotch area of the panty. In that regard, the crotch width of a typical female panty generally ranges in width from about 5.0 cm. (1.9 in.) to about 7.5 cm. (2.95 in.), although it can also be wider in some countries.

Flaps 34 and 36 serve at least two purposes. First, the flaps are interposed between the wearer's panty and the wearer's inner thighs, to avoid soiling of the wearer's body and panty by bodily exudates in liquid form. The flaps provide that benefit by forming along the edges of the leg openings of the panty a barrier to the passage of liquid. Second, the overlapped flaps help to keep the sanitary napkin properly positioned in a longitudinal direction along the crotch region of the panty. In that regard, one or both flaps are provided with suitable attachment means so that the flaps can be attached to each other adjacent the outer, or garment-facing side of the panty after the flaps have been folded around the inner edges of the leg openings of the panty and against the outside surface of the crotch region of the panty.

A number of sanitary napkin structures having side flaps suitable or adaptable for use with a sanitary napkin in accordance with the present invention are disclosed in U.S. Pat. No. 4,687,478, entitled, “Shaped Sanitary Napkin With Flaps,” which issued on Aug. 18, 1987, to Van Tilburg; in U.S. Pat. No. 4,589,876, entitled, “Sanitary Napkin,” which issued on May 20, 1986, to Van Tilburg; and in U.S. Pat. No. 4,608,047, entitled, “Sanitary Napkin Attachment Means,” which issued on Aug. 26, 1986, to Mattingly. The disclosures of each of those patents are incorporated herein by reference to the same extent as if fully rewritten.

Flaps 34 and 36 can be made in various shapes, including those shapes shown in FIGS. 1, 3, and 4, and they can be made from a variety of suitable materials, including materials similar to those employed to make topsheet 22 and backsheet 26, as well as tissue, or a combination of those materials.

In the embodiment illustrated in FIGS. 1 and 2, flaps 34 and 36 are each lateral extensions of backsheet 26 and of topsheet 22, and they are of generally trapezoidal form. Thus flaps 34 and 36 are each two-ply structures, wherein the body-contacting surface of each flap is the same soft material as that of topsheet 22 and the garment-facing surface of each flap is the same material as that of backsheet 26. Such a two-ply structure is desirable in that the body-contacting surfaces of the flaps provide a soft, compliant, non-irritating feeling that adds to the wearer's comfort. And the liquid-impervious nature of the garment-facing surfaces of the flaps provides additional protection to the wearer's clothing.

### Flap Fastening Systems

As shown in FIG. 1, each of flaps 34 and 36 carries a respective element of a suitable flap fastening system for fastening the flaps together after they have been folded over and positioned against the outermost surface of the crotch area of the panty. The fastening system includes a first fastener element 52 and a second fastener element 54, each of which can include mechanical fastening elements. As used herein, the term “mechanical fastening elements” includes fastening elements that mechanically engage each other and that also enable the flaps to be attached to each other over a range of degrees of overlap of the flaps. Such mechanical fastening elements can include any of the well-known fastening elements that achieve a variable-position attachment by mechanical engagement, such as VELCRO or other hook- and loop-type fastening elements. Such other hook-and loop-type fastening elements include those disclosed in U.S. Pat. Nos. 5,058,247, entitled, “Mechanical Fastening Prong,” which issued on Oct. 22, 1991, to Thomas et al., and in U.S. Pat. No. 5,116,563, entitled, “Process for Producing a Mechanical Fastener,” which issued on May 26, 1992, to Thomas et al. As used herein, “variable-position” fastening elements are those fastening elements that can be fastened together over a range of overlap positions of the fastener elements and without the necessity for precise positioning of each of the fastener elements relative to each other.

When a first fastener element includes a mechanical closure element, the second fastener element can include “identical” complementary mechanical fastening elements. Alternatively, the second fastener element can include “distinct” complementary mechanical fastening elements.

As used herein, the term “complementary” as applied to a fastener system refers to the components of a two-component fastener system, wherein the components are engageable with each other to effect a fastened connection therebetween that will resist inadvertent separation of the components, yet will permit manual separation of the components upon the application to the components of a sufficiently large separation force. The components can each be the same material, such as an adhesive that will adhere to itself, or they can each be different materials, such as an adhesive and a surface to which the adhesive can adhere, or such as a hook fastener element and a loop fastener element.

As used herein, the term “identical” as applied to complementary mechanical fastening elements identifies a mechanical fastening system wherein the engaging elements of the first and second fastener elements have the same configuration and have respective structures that interconnect to hold the fastener elements together. Examples of such systems are disclosed in U.S. Pat. No. 4,322,875, entitled, “Two Strip Materials Used For Forming Fasteners,” which issued on Apr. 16, 1982, to Brown, et al., the disclosure of which is hereby incorporated herein by reference to the same extent as if fully rewritten.

As used herein, the term “distinct” as applied to complementary mechanical fastening elements refers to mechanical fastening systems wherein a first fastener element is structurally different from a second fastener element, but is engageable therewith, such as a hook-based fastening material and a loop-based fastening material. For example, if the second fastener element is a loop-based fastening material, then the first fastener element will be a hook-based fastening material, and vice versa.

As used herein, the term “hook-based fastening material” identifies a material having engagement elements. Thus, the hook-based fastening material can also be referred to as a male fastener. It should also be understood that the use of the term “hook” is non-limiting in the sense that the engagement elements can be of any suitable shapes, such as those engagement elements known to those skilled in the art, so long as they are adapted to engage a complementary mechanical closure element, such as a loop-based fastening material or even another hook-based fastening material.

A hook-based fastening material is preferably adapted to mechanically engage fibrous elements of a loop-based fastening material in order to provide a secure connection between those elements. Thus, a hook-based fastening material according to the present invention can be manufactured from a wide range of materials. Suitable materials include nylon, polyester, polypropylene, or any combination of those materials or of other suitable materials that are known to those skilled in the art. A suitable hook-based fastening material includes a number of shaped engagement elements that project from a backing, such as the commercially-available material designated SCOTCHMATE Brand, No. FJ3402, available from the Minnesota Mining and Manufacturing Company, of St. Paul, Minn. The engaging elements can have any shape, such as hooks, “T's”, mushrooms, or any other effective shape that is known to those skilled in the art. A suitable hook-based fastening material is disclosed in U.S. Pat. No. 4,846,815, entitled, “Disposable Diaper Having An Improved Fastening Device,” which issued on Jul. 11, 1989, to Scripps, the disclosure of which is hereby incorporated herein by reference to the same extent as if fully rewritten.

An especially preferred hook-based fastening material includes an array of prongs that are formed of thermoplastic material. Hot melt adhesive thermoplastics, in particular polyester and polyamide hot melt adhesives, are particularly well suited for forming the prongs of the hook-based fastening material. The prongs are preferably manufactured using a modified gravure printing process by printing the thermoplastic material in its molten state onto a substrate in discrete units, severing the material in a manner that allows stretching of a portion of the thermoplastic material before severance, and allowing the stretched molten material to “freeze”, or solidify, resulting in prongs. This preferred hook-based fastening material and methods and apparatus for making such a hook-based fastening material are more fully described in European Patent Application No. 0 381 087, owned by The Procter & Gamble Company, and published on Aug. 8, 1990, the disclosure of which is hereby incorporated herein by reference to the same extent as if fully rewritten.

A loop-based fastening material generally provides a plurality of fibrous elements that can engage with the complementary engagement elements of a hook-based fastening material. The loop-based fastening element can be manufactured from a wide range of materials to provide the fibrous elements, which are preferably in the form of loops. Suitable materials that can form the loop-based fastening element include nylon, polyester, polypropylene, combinations of those materials, and other suitable materials that are known to those skilled in the art. One such suitable loop-based fastening material includes a number of fiber loops that project outwardly from a backing, such as the commercially available material designated SCOTCHMATE Brand nylon woven loop, No. SJ3401, available from the Minnesota Mining and Manufacturing Company, of St. Paul, Minn. Another commercially available loop-based fastening material includes a tricot knit fabric having a plurality of nylon filament loops projecting outwardly from a nylon backing, such as the loop-based fastening material commercially available from Guilford Mills, of Greensboro, N.C.

A suitable and relatively inexpensive loop-based fastening material and a method of making such a loop-based fastening material are described in European Patent Application No. 0 289 198, owned by The Procter & Gamble Company, and published on Nov. 2, 1988, the disclosure of which is hereby incorporated herein by reference to the same extent as if fully rewritten.

The loop-based fastening material can also be a woven or a nonwoven fabric, or any other type of suitable fibrous material or loop-containing material that is known to those skilled in the art. Examples of nonwoven materials suitable for use as a loop-based fastening material include those identified herein as useful for serving as topsheet 22 of absorbent article 20. In one preferred embodiment of the present invention the loop-based fastening material is a nonwoven material of which topsheet 22 is made.

An example of a fastening system wherein the first and second fastening elements each include mechanical closure elements including hook and loop-based fastening materials is disclosed in U.S. Pat. No. 4,869,724, entitled, “Mechanical Fastening Systems With Adhesive Tape Disposal Means For Disposable Absorbent Articles,” which issued on Sep. 26, 1989, to Scripps. Fastening systems utilizing mechanical closure elements are also disclosed in U.S. Pat. No. 4,846,815, entitled, “Disposable Diaper Having An Improved Fastening Device,” which issued on Jul. 11, 1989, to Scripps; and U.S. Pat. No. 4,894,060, entitled, “Disposable Diaper With Improved Hook Fastener Portion,” which issued on Jan. 16, 1990, to Nestegard. A primary fastening system having a combination of adhesive and mechanical closure elements is disclosed in U.S. Pat. No. 4,946,527, entitled, “Pressure-Sensitive Adhesive Fastener And Method of Making Same,” which issued on Aug. 7, 1990, to Battrell. The disclosures of each of the foregoing patents are hereby incorporated herein by reference to the same extent as if fully rewritten.

Alternatively, the fastening system can include a layer of adhesive material. Adhesives useful in the present invention are preferably pressure-sensitive adhesives formulated to adhere to a variety of types of surfaces at ambient temperature by applying only light pressure. Particularly preferred adhesives for use herein as an adhesive attachment layer are various hot-melt, pressure-sensitive adhesives known to those skilled in the art. An example of a suitable hot-melt, pressure-sensitive adhesive is a Kraton-based adhesive with tackifiers and other additives, such as that marketed by Findley Adhesives, Inc., of Elm Grove, Wis., under the trade names FINDLEY 990 or FINDLEY H-2085.

Additional examples of suitable pressure-sensitive adhesives are Century A-305-IV, manufactured and sold by Century Adhesives Corporation, of Columbus, Ohio, and Instant Lock 34-2823, manufactured and sold by National Starch & Chemical Company, of Bridgewater, N.J. Suitable adhesive fasteners are also disclosed in U.S. Pat. No. 4,917,697, entitled, “Sanitary Napkins Having Flaps and Stress Relief Means,” which issued is on Apr. 17, 1990, to Osbom, III et al., the disclosure of which was earlier incorporated herein by reference. The pressure-sensitive adhesives intended for use in connection with the present invention retain sufficient tackiness, even after an initial adhesive attachment bond is separated, to remain effective to permit a sufficiently strong reattachment bond so that the flaps can again be securely reattached to each other.

When pressure-sensitive adhesive is employed in the form of an adhesive layer carried by the absorbent article, the adhesive layer can be covered with a removable release liner strip before the article is used, to prevent the adhesive from contacting an unintended surface before use. Suitable release liner strips include those commercially available release liner strips known as BL30MG-A Silox E1/0 and BL30MG-A Silox 4P/O, manufactured and sold by Akrosil Corporation, of Menasha, Wis.

When the first fastener element is an adhesive layer, the second fastener element preferably is a release area, to enable the fastener elements to be separated and subsequently reattached, if desired by the wearer. The release area can be the backsheet itself when the adhesive is of the type that adheres to the backsheet with a sufficient holding force to hold the napkin in position, but that also permits release of the fastener connection without tearing of the backsheet and that also permits subsequent reattachment of the adhesive to the backsheet with a sufficient holding force to hold the napkin in position.

Whether an adhesive and backsheet arrangement can be successfully utilized is dependent upon the nature of the backsheet material, its tensile strength, the nature of its surface, and the force necessary to separate the adhesive from the backsheet. If the backsheet is thin and could possibly be torn by the removal of the adhesive first fastener element from the backsheet, then preferably the second fastener element is a discrete, separate release layer or release coating that is securely joined to the backsheet, such as by a strong adhesive bond. The release layer or coating preferably has a surface that enables the adhesive fastener element to securely, yet releasably, adhere to, to be removed from, and to re-adhere to the release layer with sufficient tenacity to provide a good bond after several attachments and removals of the fastener elements.

The fastening system elements can further include combinations of adhesive and mechanical fastener elements. For example, the fastening system elements can include a combination fastener, such as a hook-based fastening material and an adhesive attachment layer juxtaposed with the hook-based fastening material, or it can include a mechanical closure element, such as a hook-based fastening material having a layer of adhesive coated over a portion of the hook-based fastening material. An example of a fastener having a combination of a mechanical element and an adhesive element is a pressure-sensitive adhesive fastener having a textured fastening surface, such as that disclosed in U.S. Pat. No. 4,946,527, entitled, “Pressure-Sensitive Adhesive Fastener And Method of Making Same,” which issued on Aug. 7, 1990, to Battrell, the disclosure of which is hereby incorporated herein by reference to the same extent as if fully rewritten.

A fastening system for providing a variable attachment position fastener capable of being passively activated can alternatively further include any cohesive materials of the type that are well known to those skilled in the art. For example, one form of cohesive material can be provided in strip form as foams, rubbers, such as crepe or latex rubbers, other adhesives, or a high-static-charge vinyl material. A separable fastener of a high static vinyl material is more fully described in U.S. Pat. No. 4,979,613, entitled, “Separable Fastening Device,” which issued on Dec. 25, 1990, to McLaughlin et al., the disclosure of which is hereby incorporated herein by reference to the same extent as if fully rewritten.

The fastening system elements can alternatively include a separate element joined to absorbent article 20, or it can be a unitary element joined with one of the other elements of the article. For example, topsheet 22 can be made from a material, such as a nonwoven web, that is capable of mechanically engaging the other attachment element, which can be a hook-based fastening material. Additionally, backsheet 26 can be formed from a web having a textured pattern and having a layer of adhesive coated over a portion of the garment-facing surface of the backsheet to form a combination fastener of mechanical and adhesive elements, such as is shown in the hereinbefore-referenced U.S. Pat. No. 4,946,527, which issued to Battrell. In each of those cases, the attachment element is unitary with another element of the absorbent article. Alternatively, the attachment element can include a discrete strip or patch joined to the absorbent article.

In one preferred embodiment the first attachment element includes a discrete patch of a hook-based fastening material joined to the backsheet to form a portion of the outer surface of the backsheet, while the second attachment element is a unitary element forming a portion of the topsheet 22.

### Positioning of the Napkin for Use

In use, the absorbent article can be held in a desired position relative to the body of a wearer by any known holding or support means. Preferably, however, when the absorbent article is in the form of a sanitary napkin 20, as shown in FIGS. 1 and 2, napkin 20 is placed within the wearer's undergarment, or panty, on the interior surface at the crotch area of the panty, and is retained in that position by a suitable fastening arrangement, such as an adhesive carried on the outwardly-facing surface of the backsheet of the napkin.

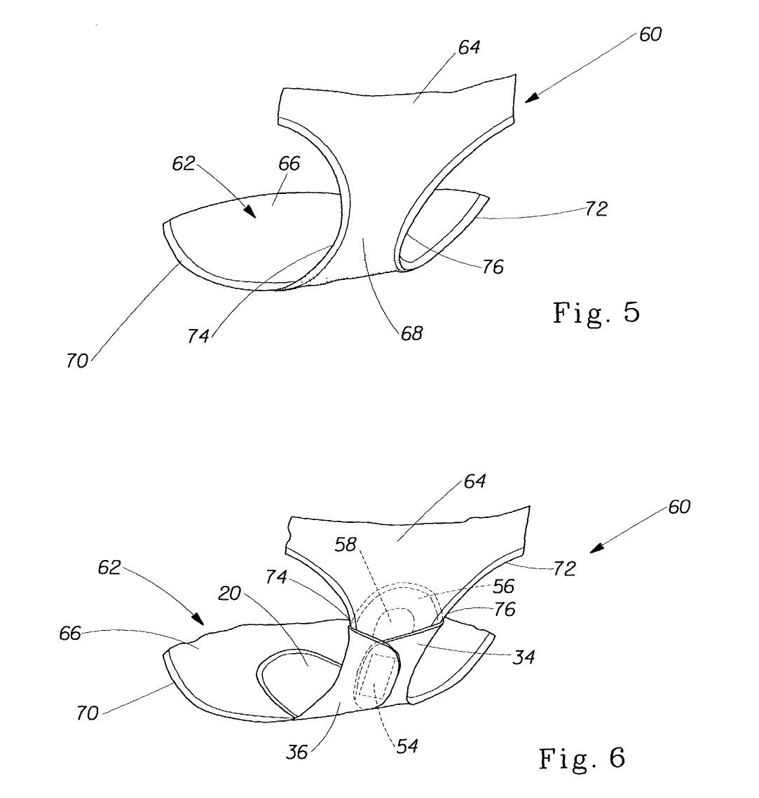

FIG. 5 is a fragmentary perspective view of a portion of a panty 60 showing the crotch area 62. Panty 60 includes a front panel 64 and a rear panel 66, between each of which extends a narrow, elongated, crotch panel 68 that interconnects front and rear panels 64, 66. Additionally, panty 60 includes a right leg opening 70 and a left leg opening 72. Right leg opening 70 defines a right side crotch edge 74 and left leg opening 72 defines a left side crotch edge 76. FIG. 6 shows panty 60 with a napkin 20 in its operative position within crotch area 62 of the panty.

An adhesive carried by sanitary napkin 20 permits convenient and effective attachment of napkin 20 to the interior of panty 60 at crotch area 62. Thus, as more clearly seen in FIG. 2, at least a portion of the outer, garment-facing surface 56 of backsheet 26 is preferably coated with a suitable adhesive layer 58. Any adhesive or glue known to those skilled in the art can be used to attach napkin 20 to the interior of panty 60 at crotch area 62, but pressure-sensitive adhesives are preferred. Suitable pressure-sensitive adhesives include CENTURY A-305-IV, manufactured by the Century Adhesives Corporation, of Columbus, Ohio, and INSTANT LOCK 34-2823, manufactured by the National Starch and Chemical Company, of Bridgewater, N.J. Other suitable adhesive fastening arrangements are disclosed in U.S. Pat. No. 4,917,697, entitled, “Sanitary Napkins Having Flaps and Stress Relief Means,” which issued on Apr. 17, 1990, to Osbom, III et al., the disclosure of which is hereby incorporated by reference to the same extent as if fully rewritten.

Preferably, a removable release liner strip 59 is placed against and in overlying, covering relationship with adhesive layer 58 before use of the sanitary napkin. Release liner strip 59 serves to keep adhesive layer 58 from drying out, to keep it clean, and to prevent it from adhering to a surface other than the crotch portion of the panty before the napkin is positioned for use. Suitable release liner strips include those commercially available release liner strips known as BL30MG-A Silox E1/0 and BL30MG-A Silox 4P/O, manufactured and sold by the Akrosil Corporation, of Menasha, Wis. Other suitable release liners are disclosed in the hereinabove-referenced U.S. Pat. No. 4,917,697.

Sanitary napkin 20 is positioned for use by removing release liner strip 59 and thereafter placing the napkin against the interior surface at crotch area 62 of panty 60, in the position shown generally in FIG. 6, so that adhesive layer 58 contacts the interior surface of the panty at crotch area 62 and adheres to it to maintain the napkin in the desired position within the panty. During positioning of the napkin within the panty, side flaps 34 and 36 are released from any interconnections therebetween and from any separate interconnecting members and are allowed to hang downwardly from napkin 20 and to extend into respective leg openings 72 and 70. When so disposed, side flaps 34, 36 each overlie respective crotch edges 76, 74 to cover those edges with liquid-impervious backsheet 26 and thereby protect them from contact with liquids that are carried by absorbent core 24.

The panty with the napkin in position in the crotch area is then put on in the usual manner, so that the wearer's legs extend into the respective leg openings, and the panty is drawn upwardly into normal wearing position so that napkin 20 is positioned adjacent the vaginal cleft of the wearer. The position of napkin 20 can be adjusted, if necessary or desired, by separating the adhesive bond holding napkin 20 to the interior surface of crotch panel 68, repositioning napkin 20 relative to crotch panel 68, and then again pressing napkin 20 against the inner surface of crotch panel 68 to re-establish an adhesive bond therebetween.

After napkin 20 has been properly positioned for most effective absorption and comfort, any release strips carried by fastener elements 52 and 54 that were not previously removed are then removed. Flap 34 is first placed against the outer surface of crotch panel 68 so that first fastener element 52 faces outwardly, and then flap 36 is placed against the outermost surface of flap 34, so that fastener elements 52 and 54 are in intimate contact with each other. Napkin 20 is then securely positioned within panty 60 in its preferred operative position.

If after napkin 20 is positioned, or if after being worn for a time its position relative to panty 60 or relative to the wearer's vaginal cleft should be incorrect for effective absorption of liquids, or should the napkin shift from its previous, preferred position, possibly because it was not properly positioned initially, the napkin can easily be repositioned and reattached. To do so the flaps are disconnected from each other, the adhesive area 58 of the napkin is disconnected from the interior surface of the panty at the crotch area, and napkin 20 is repositioned to a desired new position. Thereafter, adhesive layer 58 is again pressed against the interior surface of crotch panel 68 so that adhesive layer 58 again contacts the interior surface of the panty at crotch area 62 and adheres to it to maintain the napkin in the desired new position within the panty. The flaps are then reattached in the same manner as they were previously. Because the fastener elements are such that secure reattachment can effectively be made, the reattached napkin will continue to function properly and will remain in its new position.

### Description of Alternative Fastener Element Positions

In the embodiment illustrated in FIGS. 1 and 2, first and second fastener elements 52 and 54 are generally rectangularly-shaped and each fastener element is positioned on a different major surface of napkin 20. First fastener element 52 carried on flap 34 is positioned on the body-facing surface of topsheet 22, whereas second fastener element 54 carried on flap 36 is positioned on garment-facing surface 56 of backsheet 26. And as shown in FIG. 1, each of fastener elements 52, 54 is of elongated form and each has a longitudinal axis that is generally parallel with longitudinal axis 38 of absorbent core 24.

Fastener element 52 can be in the form of a securement member that can be a layer of pressure sensitive adhesive, the hook or loop element of a hook-and loop-type fastening system, or a piece of double-sided adhesive tape carried on flap 34. When in the form of a double-sided adhesive tape, fastener element 52 has a pressure-sensitive adhesive coating on its outwardly-facing surface and preferably it includes a release cover strip 53 that overlies and protects the adhesive coating before use of the napkin. Similarly, when fastener element 52 is in the form of a layer of pressure-sensitive adhesive a release cover strip can also be included to cover and protect the adhesive layer. Fastener element 52 can have a longitudinal length of the order of from about ½ inch to about 3 inches, and a transverse width of the order of from about ¼ inch to about 1 inch. The length and width of fastener element 52 will be influenced by the shape of the flap, as well as by the lateral position of the fastener element relative to napkin longitudinal centerline 38, in order for the fastener elements 52 and 54 to be properly positioned so they come into at least partial surface contact with each other when the flaps are brought into overlapping relationship.

Second fastener element 54 can be in the form of a contact surface that can be the complementary portion of a hook and loop fastener system, or it can be a release area that allows attachment, release, and reattachment of a pressure-sensitive-adhesive-bearing surface. As with first fastener element 52, second fastener element 54 can be of elongated form and can have a length and a width that is influenced by the shape of the flap, as well as by the lateral position of the fastener element relative to napkin longitudinal centerline 38. In that regard, the length of second fastener element 54 can be from about ½ inch to about 3 inches, and the width can be from about ¼ inch to about 1 inch. Alternatively, second fastener element 54 can be garment-facing surface 56 of backsheet 26, without any additional elements, provided that first fastener element 52 can securely and releasably be connected with that backsheet surface.

The lateral spacing of first and second fastener elements 52 and 54 relative to longitudinal centerline 38 is dependent, in part, upon the range of panty crotch widths. As noted earlier herein, the panty crotch width can vary; it is generally dependent upon the size of the panty. Consequently, the spacing between the fastener elements can be of the order of from about 4 inches to about 6½ inches. However, if the backsheet surface 56 is itself the second fastener element, then it is not critical where the first fastener element is positioned, so long as it is in a position to contact the outermost surface of flap 36 when each of the flaps has been folded over the respective edges at the crotch area of the panty. Moreover, it will be apparent that the connection of the flaps of this napkin embodiment, when the napkin is to be connected with a panty, must be effected by following a particular flap folding sequence—specifically, flap 34 must be folded over the outer surface of the crotch area of the panty before flap 36, so that first fastener element 52 is facing the surface of flap 36 to enable a secure connection to be effected between the two flaps.

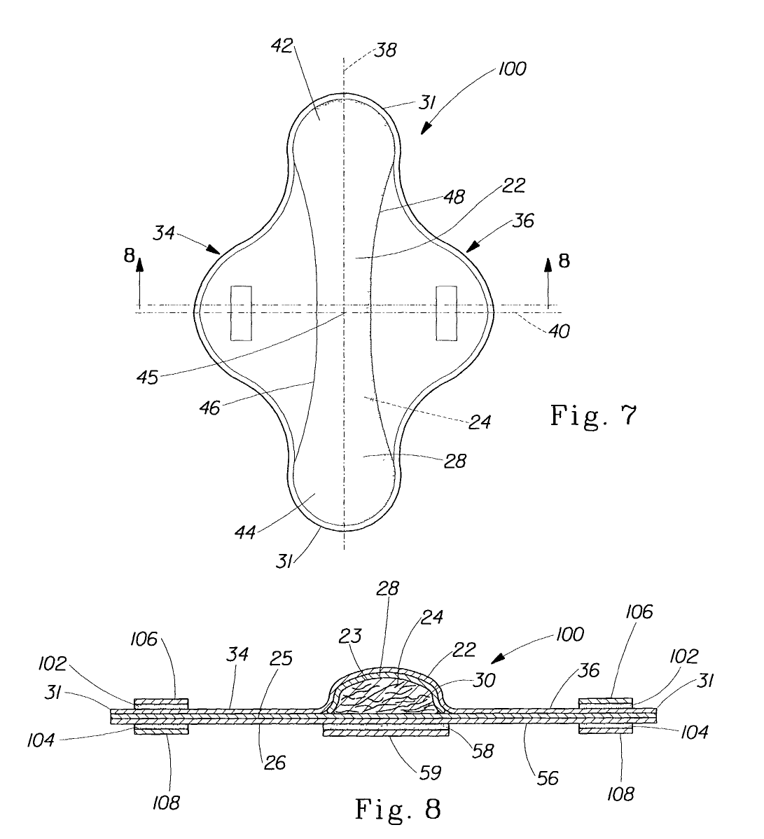

In another embodiment of the present invention as illustrated in FIGS. 7 and 8, each of flaps 34, 36 of napkin 100 carries two fastener elements 102, 104. The fastener elements carried by a particular flap can be the same type of fastener element, or, as shown in FIGS. 7 and 8, they can be complementary elements. In any event, one fastener element is positioned on the body-facing surface of one of the flaps, and a complementary or cooperating fastener element is positioned on the opposite, or garment-facing surface of the other flap. This embodiment of the invention permits either of the flaps to be folded over first and then connected with the other flap in at least partially overlapping relationship. It thereby simplifies the flap attachment process by eliminating the need for the wearer to fold the flaps in a particular sequence, as is necessary to utilize properly and effectively the embodiment of the invention illustrated in FIGS. 1 and 2. Again, the fastener elements employed can be any of the complementary elements of the fastening systems hereinbefore described.

As shown in FIGS. 7 and 8, both fastener elements carried by each flap are aligned with each other and are coextensive with each other. The respective fastener elements can be of any desired size, shape, and position, so long as the flaps can be connected together and retained in the desired overlapped position by the interconnected fastener elements of the respective flaps. As also shown in FIGS. 7 and 8, fastener elements 102 and 104 are pressure-sensitive adhesive areas that can be covered by respective release strips 106, 108. As earlier noted, however, other forms of complementary fastener elements can also be employed.

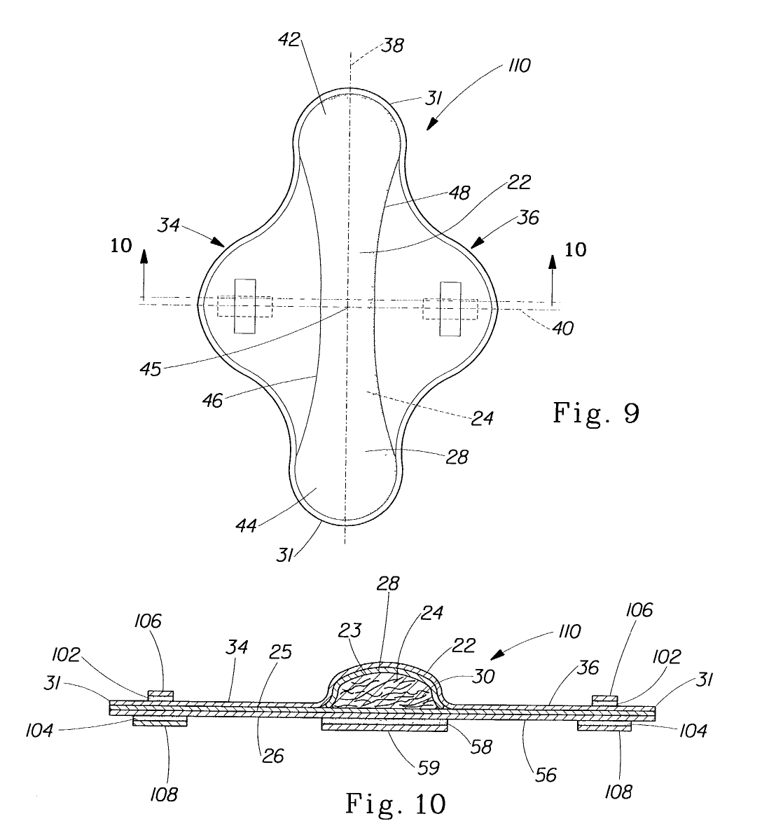

A further embodiment of the present invention is shown in FIGS. 9 and 10. In that embodiment the fastener elements 102, 104 of napkin 110 are disposed on both flap surfaces, as in the embodiment shown in FIGS. 7 and 8. However, in the FIGS. 9 and 10 embodiment the elongated first and second fastener elements 102, 104 are not coextensive with each other, but, instead, are angularly rotated with respect to each other, in this instance by an angle of substantially 90°. That positioning arrangement enables flaps 34, 36 to be readily fastened together without the need for carefully aligning the centerlines of the respective flaps relative to each other to ensure overlap of the respective complementary fastening system elements. As will be appreciated by those skilled in the art, the angular relationship between the fastener elements carried on a particular flap can be different from 90°, if desired, so long as the fastener elements are angularly disposed relative to each other. The angular relationship of the fastener elements facilitates flap attachment and permits attachment of the flaps to each other even if the complementary fastener elements carried by the flaps are slightly offset from each other after the flaps have been folded over the edges of the crotch area of the panty.

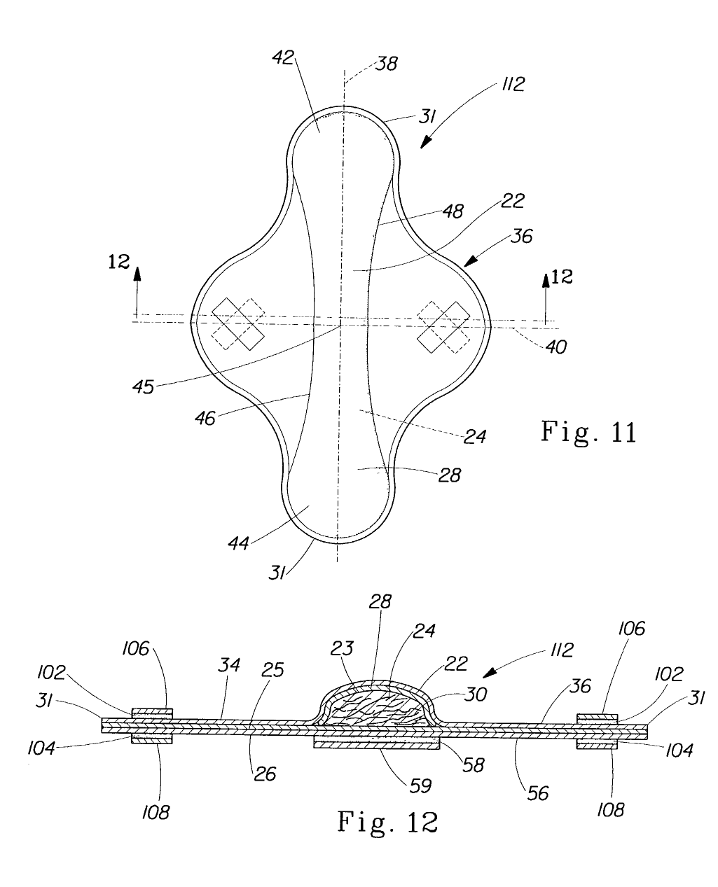

A still further embodiment of the present invention is illustrated in FIGS. 11 and 12, in which the fastening system elements 102, 104 of napkin 112 are disposed with respect to each other in the same relative angular arrangement as shown in FIG. 9, except that the axes of each of the fastener elements has been rotated by 45° relative to their positions in the FIG. 9 embodiment. As was the case with the FIG. 9 embodiment, the orientation of the fastener elements in the FIG. 11 embodiment enables connection of the flaps with each other without the need to carefully align the centerlines of the flaps or to carefully align the fastener elements, because the orientation of the fastener elements provides a greater opportunity for at least partial overlap of the respective fastener elements for a large number of possible relative positions of the flaps.

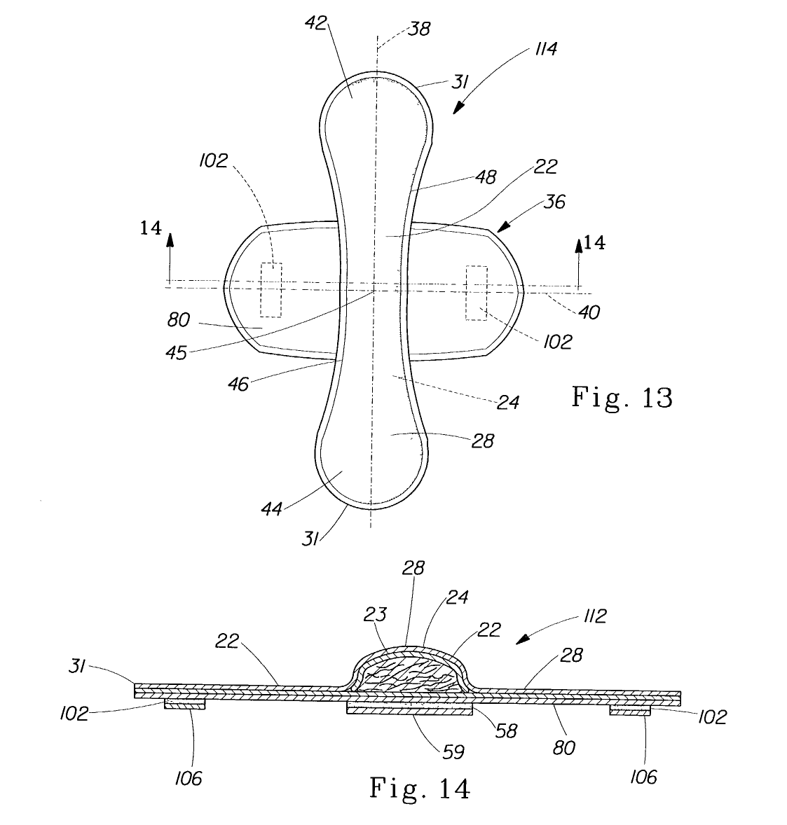

FIGS. 13 through 16 show napkin structures wherein the flaps are provided by one or more separate and distinct, laterally extending members that are attached to the backsheet of the napkin. Moreover, as shown, the separate flap members can have their longitudinal axes aligned with transverse axis 40 of the absorbent core, if desired. However, that position is not essential for satisfactory operation of those embodiments, and the flap members can be positioned at any desired and convenient position along the longitudinal centerline of the absorbent core of the napkin.

In the embodiment shown in FIGS. 13 and 14, a non-integrally-formed, separate, unitary piece of flap material 80 extends across napkin 114, on the backsheet side thereof. Flap material piece 80 defines each of the respective flaps, and it can be made from any of a number of materials, including materials that are different from the materials utilized to make the backsheet. For example, suitable materials for that purpose include those disclosed in U.S. Pat. No. 5,558,663, entitled, “Absorbent Articles Having Undergarment Covering Components with Zones of Extensibility,” which issued on Sep. 24, 1996, to Weinberger et al., the disclosure of which is hereby incorporated by reference to the same extent as if fully rewritten. And by “non-integrally-formed” is meant that the flaps are not extensions of the backsheet material.

The use of a separate piece of material for the flaps provides distinct advantages. First of all, non-integral flaps permit a much wider range of possible flap materials to be utilized to form the flaps. Such possible flap materials can be of a type that provide greater comfort, more softness, or that have a lower cost than integrally-formed flaps that are extensions of the backsheet and topsheet materials. Secondly, non-integral flaps permit more freedom in the manufacturing process in that the same production line can readily be utilized to manufacture sanitary napkins that do not have flaps, and then by adding a suitable flap application apparatus to attach the separate piece of material the same production line can be utilized to manufacture sanitary napkins having a pair of flaps defined by the separate piece of material.

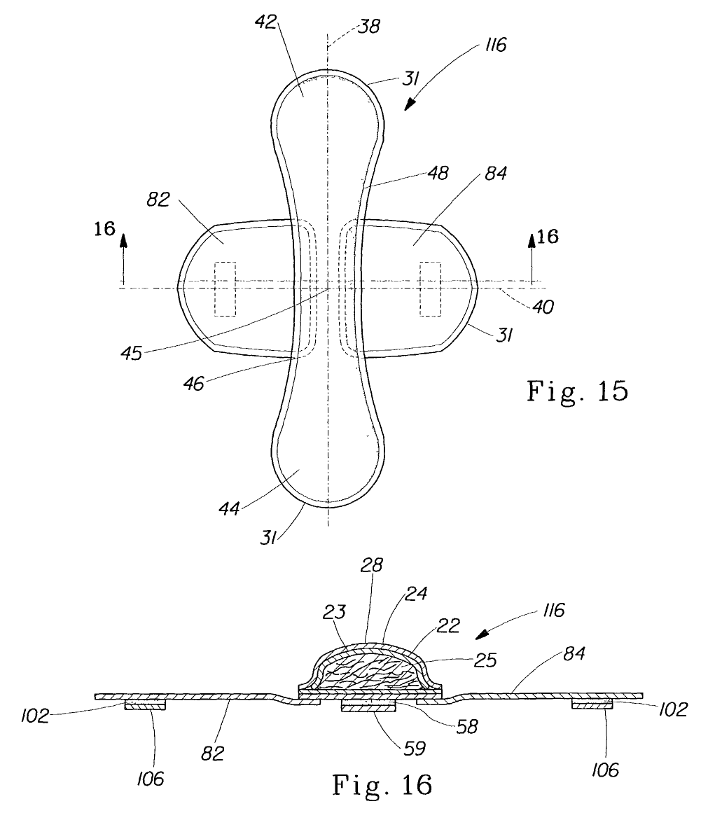

In the embodiment illustrated in FIGS. 15 and 16, two discrete flap members 82 and 84 are provided, one extending outwardly from each side of napkin 116. Flaps 82 and 84 can be made of the same materials as were identified above in the context of unitary flap member 80 shown in FIGS. 13 and 14 and they can be secured to backsheet 26 in the same manner. In the FIGS. 15 and 16 embodiment, however, each of flaps 82, 84 is individually formed and is secured to garment-facing surface 56 of backsheet 26 at respective positions that lie laterally outwardly of napkin longitudinal centerline 38.

### Napkins Having Zones of Flap Extensibility

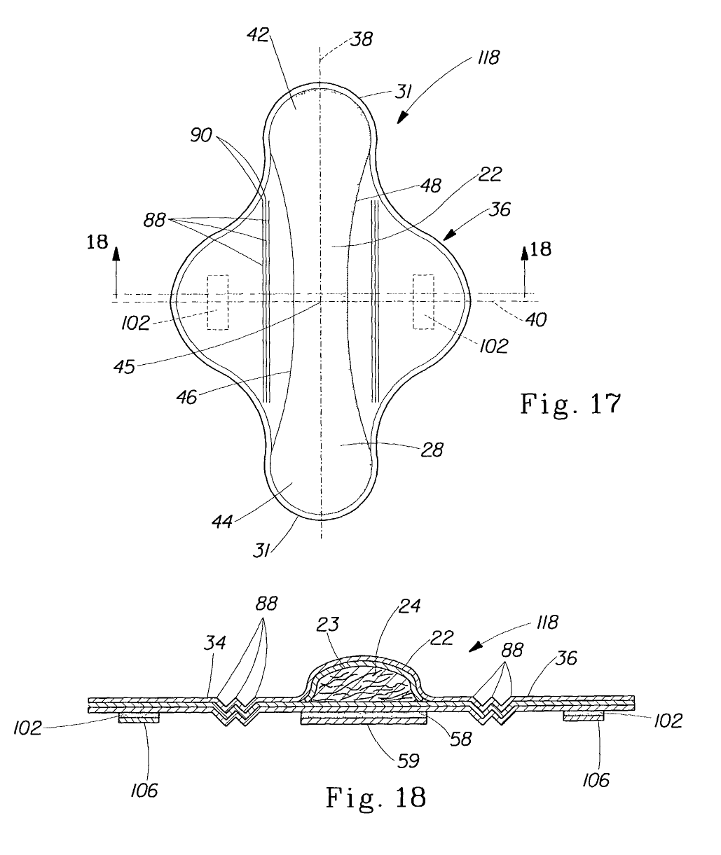

Further embodiments of the present invention are illustrated in FIGS. 17 through 23. In each of those further embodiments lateral extensibility has been imparted to discrete portions of each of the flaps, to permit a limited amount of lateral stretch of the flap material at those particular portions of the flaps, to respond to and to accommodate particular stresses imposed on the flaps when the napkin is placed in its desired operative position in the crotch area of a panty, or stresses applied to the flaps while the napkin is worn and is in use.

When positioned within the crotch area of a panty and worn, a sanitary napkin assumes generally the same U-shape that the panty crotch assumes, in a longitudinal direction of the panty crotch and napkin, because of the conformation of the adjacent anatomical surfaces of the body of the wearer. Similarly, the flaps and the joinder zones between and interconnecting the flaps with the main portion of the backsheet of the napkin also tend to assume what is generally a U-shape in the longitudinal direction of the napkin. Consequently, when the flaps are folded over respective U-shaped crotch edges 74 and 76 (see FIGS. 5 and 6), the U-shaped fold line of the napkin, when viewed in a plane parallel with the longitudinal axis of the absorbent core, and the forces applied to the flaps during the flap folding and attachment operations, impose upon the flaps tensile stresses that are greatest at the longitudinal extremities of each of the fold lines.

One approach to relieving the stresses in those areas of sanitary napkins having flaps involves the formation of V-shaped notches adjacent the flap fold lines, as disclosed in U.S. Pat. No. 4,917,697, entitled, “Sanitary Napkins Having Flaps and Stress Relief Means,” which was previously referred to herein and the disclosure of which was earlier incorporated herein by reference.

In the embodiment shown in FIGS. 17 and 18, napkin 118 includes a plurality of discrete, spaced, longitudinally extending ridges 88 and grooves 90 that have been formed in each of the flaps at positions laterally outward from absorbent core 24, and along the general area of the napkin that when in use would include or be adjacent to a flap fold line. As shown, ridges 88 are of substantially equal length and are spaced laterally from each other by intervening grooves 90. The ridges and grooves are also generally parallel to each other and are substantially parallel to longitudinal axis 38 of absorbent core 24. As also shown in FIG. 17, a groove 90 extends between adjacent parallel ridges 88, so that in cross section, as shown in FIG. 18, the alternating ridges 88 and grooves 90 define an area of pleats, corrugations, or wavy areas in each of the flaps.

Formation of the ridges and grooves can be effected by embossing, or by employing similar techniques for deforming the flap material to form such ridge and groove elements. The shapes of suitable ridges and grooves, and methods and apparatus for forming them, are disclosed in U.S. Pat. No. 5,518,801, entitled, “Web Materials Exhibiting Elastic-Like Behavior,” which issued on May 21, 1996, to Chappell et al.; U.S. Pat. No. 5,156,793, entitled, “Method for Incrementally Stretching Zero Strain Stretch Laminate Web in a Non-Uniform Manner to Impart a Varying Degree of Elasticity Thereto,” which issued on Oct. 20, 1992, to Buell et al.; and U.S. Pat. No. 5,167,897, entitled, “Method for Incrementally Stretching a Zero Strain Stretch Laminate Web to Impart Elasticity Thereto,” which issued on Dec. 1, 1992, to Weber et al. The disclosure of each of those patents is hereby incorporated herein by reference to the same extent as if fully rewritten.

The formation of a localized pattern of linear ridges and grooves in the positions illustrated in FIG. 17 permits each of the flaps to be laterally elastically extensible, relative to absorbent core 24, for a limited distance. The degree of extensibility is dependent upon the nature of the flap material and the configuration and spacing of the ridges and grooves. The sizes and spacings of the ridges can be as disclosed in published PCT application No. PCT/US95/01472, entitled, “Absorbent Articles,” published on Aug. 10, 1995, as International Publication No. WO 95/20931, the disclosure of which is hereby incorporated by reference to the same extent as if fully rewritten.

Because ridges 88 and grooves 90 extend in the longitudinal direction of the napkin and along the entire longitudinal length of the flaps, portions of the flaps can be extended different distances along the entire lengths of the ridges and grooves, and need not be uniform in the direction of napkin transverse axis 40. Instead, the extension forces can be applied to a flap at an acute angle relative to transverse axis 40, in order to accommodate different levels of stress that could be imposed upon different portions of the flaps when the napkin is being connected with a panty and when the napkin is in use.

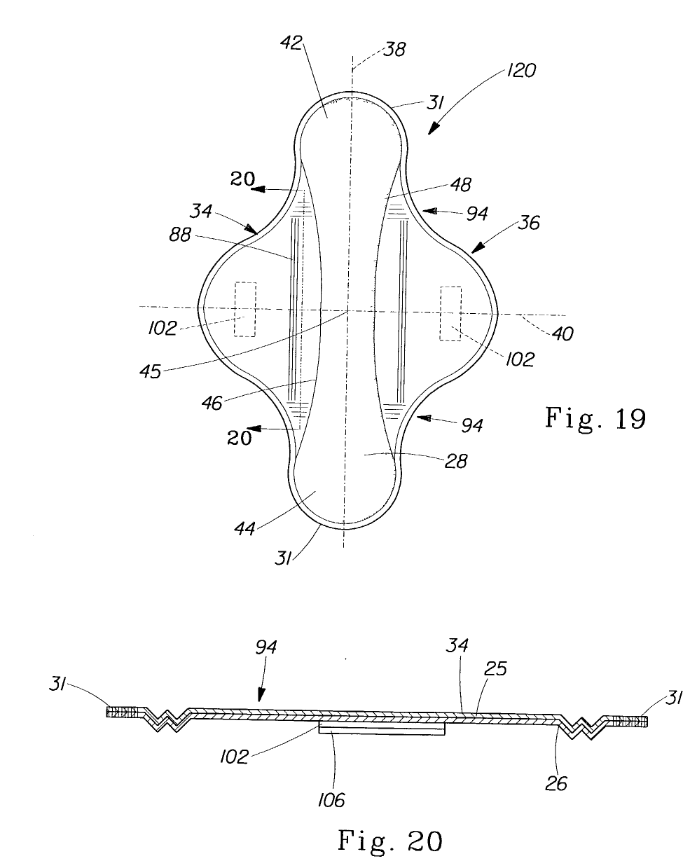

Ridges 88 and grooves 90 can also be so formed as to extend for a distance less than the entire flap longitudinal dimension, as shown in FIGS. 19, 21, and 23. In that regard, the longitudinally-positioned ridges and grooves enable the flaps to be stretched or extended laterally relative to the napkin and as may be necessary to accommodate the particular crotch width of a panty, from wide to narrow, and to still be capable of being positioned in overlapped relationship on the outer surface of the crotch area of the panty, regardless of the crotch width of the panty.

In the embodiment illustrated in FIGS. 19 and 20, napkin 120 is structurally similar to that of FIG. 17, but it includes additional zones of extensibility 94 that are formed in small, discrete portions of each of the respective flaps 34, 36 of napkin 120. Zones of extensibility 94 are of generally triangular shape, and include parallel ridges and grooves that are disposed so that their respective centerlines extend in a direction substantially perpendicular to longitudinal axis 38 of absorbent core 24. As shown, the lengths of the respective ridges increase in a direction from ends 42, 44 of napkin 20 toward transverse axis 40 of absorbent core 24.

The ridges and grooves defining extensibility zones 94 shown in FIG. 19 can have a cross-sectional appearance similar to those of ridges 88 and grooves 90 of FIG. 17, to permit longitudinal extension of the flap material in the localized areas encompassed by those zones, to enable the flaps to respond to and to accommodate stresses in different directions that can be imposed on the flaps as a result of particular conditions during installation and use of the napkin. In that regard, extensibility zones 94 permit a limited degree of extensibility in a longitudinal direction of the napkin and serve to provide stress relief points at the intersection of the longitudinal extremities of the flaps and backsheet 26. Extensibility zones 94 are directed to providing a type of stress relief that is similar to that disclosed in U.S. Pat. No. 4,917,697, which issued to Osborn, III et al., and which was identified and referred to earlier herein, in which stress-relief notches are provided at the respective longitudinal ends of each of the flaps.

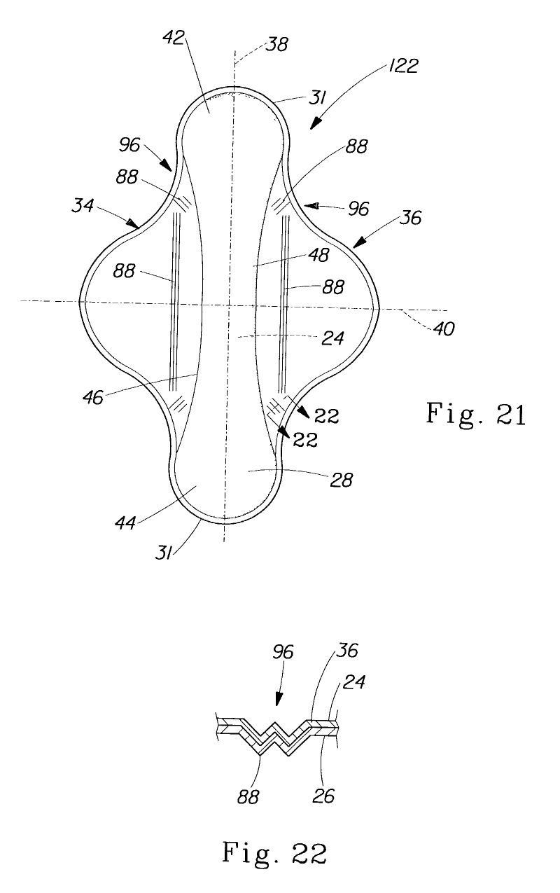

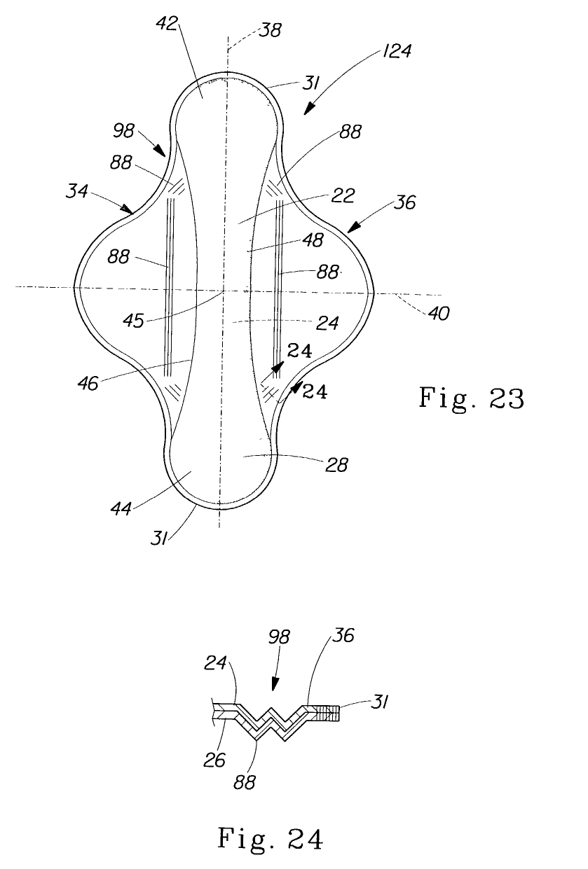

Further embodiments of a napkin having lateral extensibility and localized, angularly disposed extensibility zones are shown in FIGS. 21 through 24. Napkin 122 of FIG. 21 and napkin 124 of FIG. 23 each can have the same basic, overall structure as that of napkin 118 shown in FIG. 17.

Extensibility zones 96 positioned as shown in FIGS. 21 and 22 are generally structurally similar to extensibility zones 94 shown in FIG. 19, in that they include a plurality of parallel, spaced ridges and grooves. However, extensibility zones 96 include ridges and grooves that are differently-oriented from those of extensibility zones 94 shown in FIG. 19. The FIG. 21 extensibility zones are provided adjacent the edge of the napkin at substantially the junctions between the longitudinal ends of the flaps and the body of the napkin similar to the general positions of the extensibility zones shown in FIG. 19. In the FIG. 21 embodiment, however, the ridges and grooves are of substantially the same length, and the centerlines of the respective ridges and grooves are disposed at an acute angle relative both to longitudinal axis 38 as well as to transverse axis 40.

In the napkin embodiment shown in FIGS. 23 and 24, extensibility zones 98 are of generally triangular form and are defined by a plurality of spaced, parallel ridges and grooves. The ridges and grooves are disposed at an acute angle relative to the napkin longitudinal and transverse axes. The disposition and lengths of the ridges and grooves in extensibility zones 98 are such that the ridges and grooves are longer adjacent outer edge 31 of napkin 124, and they gradually diminish in length in an inward direction that is perpendicular to their respective centerlines. As shown, the ridges and grooves define zones of extensibility 98 in the form of isosceles triangles, although other generally triangular forms of zones of extensibility having the ridge centerlines oriented as shown in FIG. 23 can also be employed.

### Methods of Folding the Napkins

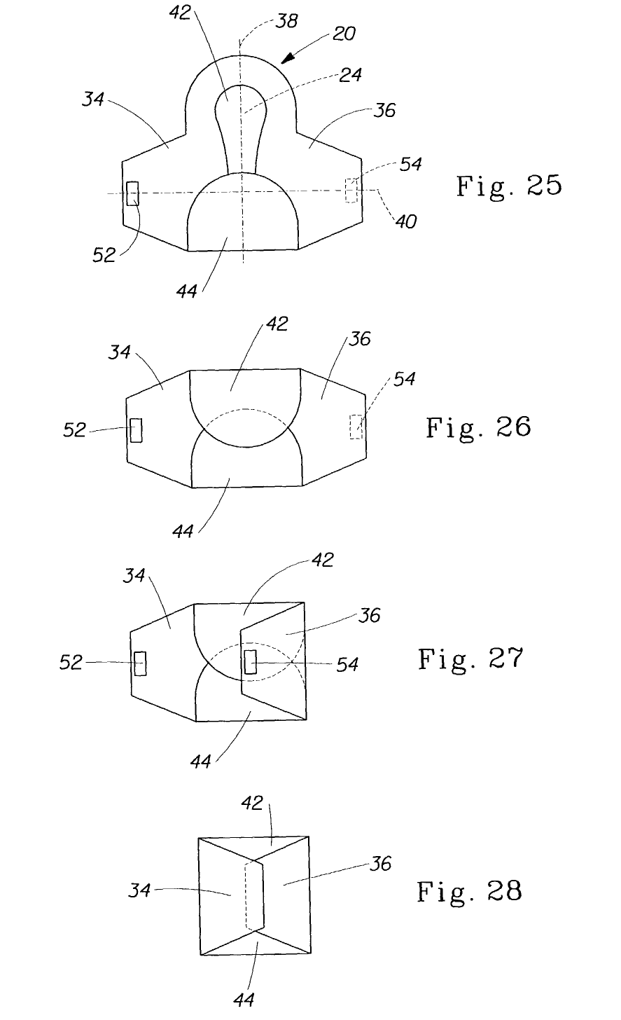

Because of the releasability and reattachability attributes of the flap fastening systems that preferably are utilized in connection with the present invention, the flaps and the fastening system can advantageously be used to hold the napkin in folded form. Before use, folding the napkin to a smaller, more compact size permits more convenient handling of the napkin for packaging purposes prior to sale, and it enables the user to carry one or more in her purse, if desired. After use, the napkin can be refolded to permit convenient and sanitary disposal of the used napkin. One form of folding technique and sequence for a napkin having the flap and fastening system structure hereinbefore described, as illustrated in FIGS. 1 and 2, is shown sequentially in FIGS. 25 through 28, in which napkin 20 in accordance with the present invention is folded in a manner to provide a more compact, generally rectangular structure that is more convenient to package and to carry.

In FIG. 25, napkin 20 is shown after the completion of the first step in the folding sequence, after end 44 has been folded inwardly along any convenient fold line toward transverse axis 40 to overlie absorbent core 24. The fold line can be any convenient transversely extending line that lies between the outer edge of end 44 and transverse axis 40, but preferably it is so selected as to cause at least a portion of end 44 to lie between flaps 34 and 36.

As the second step in the folding sequence, end 42 of napkin 20 is folded inwardly to overlie absorbent core 24 by folding about any convenient fold line that is lies between the outermost edge of end 42 and transverse axis 40. Again, the fold line is so selected as to cause at least a portion of end 42 to lie between flaps 34 and 36. As shown in FIG. 26, which shows the napkin after the completion of the second step of the folding sequence, the respective ends 42, 44 of napkin 20, when folded inwardly onto absorbent core 24, are in partially overlapped relationship with end 42 uppermost. However, if desired, the flap fold lines can be so selected that the outermost edges of respective ends 42, 44 of the napkin are spaced from each other after both ends have been folded, or the fold lines can be so selected that the outermost edges of respective ends 42, 44 are in contacting relationship, rather than in overlapping relationship. The latter two positions of the folded-over napkin ends, with ends 42 and 44 in non-overlapped relationship, serve to reduce the overall thickness of the completely folded napkin, as compared with the thickness of the folded napkin having ends that partially overlap when folded inwardly.

As shown in FIG. 27, the next step in the folding sequence is for flap 36 to be is folded inwardly over the already inwardly-folded ends 42, 44. Thereafter, flap 34 is folded inwardly over each of the already inwardly-folded ends 42, 44 and also partially over flap 36, so that fastener elements 52 and 54 engage each other, to thereby hold the folded-over portions of the napkin in their respective folded-over positions and to provide the completed folded napkin as shown in FIG. 28. The flap fastening system hereinbefore described in the context of the invention as it is shown in FIGS. 1 and 2 is utilized in the folding operation to secure the two flaps together over the inwardly-folded ends 42, 44, and thereby provide a closed, self-contained napkin that is of a more convenient size for a user to carry in her purse than if the napkin were in its flat, opened form as it is shown in FIGS. 1 and 2.

When napkin 20 is in its folded condition as shown in FIG. 28, each of the outwardly facing surfaces of the folded napkin is a portion of the garment-facing surface 56 of backsheet 26 or of the garment-facing surfaces of flaps 34, 36, so that each of the body-facing surfaces of the napkin is within the interior of the folded article, and is not exposed, to maintain the cleanliness of the absorbent portion of the napkin before use, as well as to maintain cleanliness after use and after refolding, to enable convenient carrying of the used napkin until proper disposal can be effected.

### Additional Embodiments

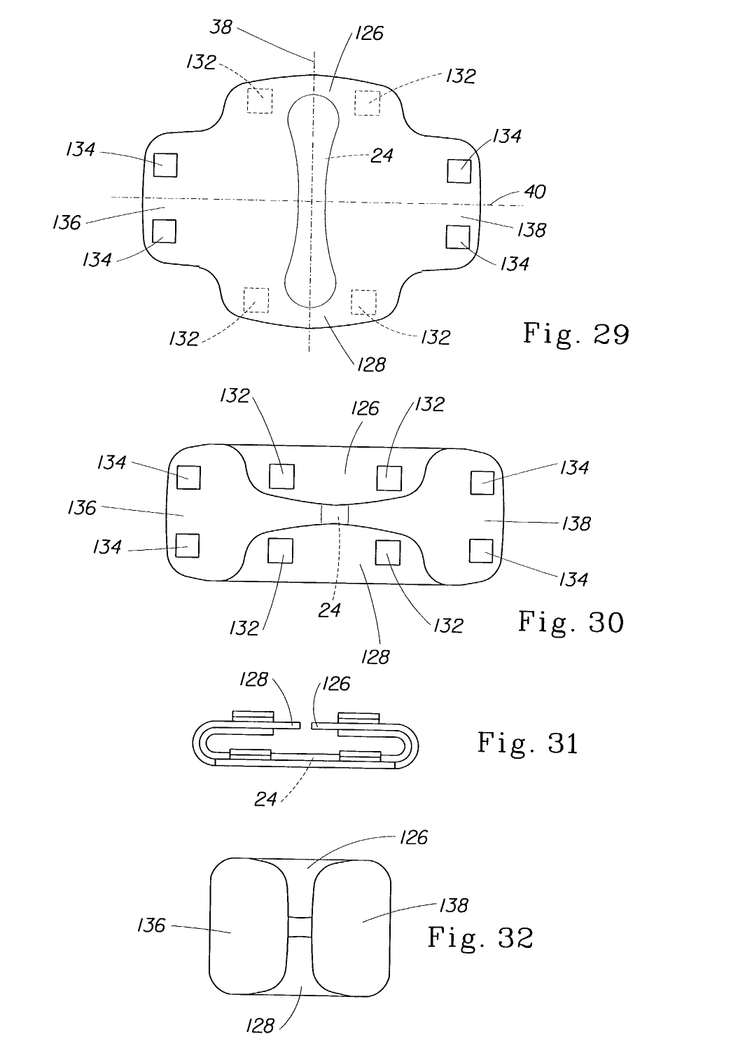

Another embodiment of the present invention to facilitate folding of the napkin into a convenient form both before and after use is shown in FIGS. 29 through 32. In that embodiment, the garment-facing surface at each of ends 126 and 128 of napkin 130 includes a pair of first fastener elements 132 of a fastening system, with a fastener element disposed on each side of longitudinal axis 38 of absorbent core 24 at each end of napkin 130. A cooperating pair of complementary, second fastener elements 134 is provided on each of side flaps 136, 138, on the respective body-facing surfaces thereof Second fastener elements 134 on each flap are disposed in spaced relationship with each other and on each side of transverse axis 40 of absorbent core 24, as shown in FIG. 29. This embodiment is adapted to be in a folded condition before use, with no fastening elements exposed, and therefore it does not require separate release strips to cover adhesive-containing areas. Consequently, this embodiment when in folded condition exposes no sticky surfaces, requires less adhesive, and provides cost savings because of the reduced quantity of adhesive needed and also because of the elimination of separate release strips.

The folding sequence for napkin 130 shown in FIG. 29 is similar to that for napkin 20 of FIGS. 1 and 2 as shown in FIGS. 25 through 28. The folding sequence includes the steps of sequentially folding inwardly each of ends 126, 128 of napkin 130 about fold lines that extend generally parallel to transverse axis 40, so that the ends each overlie absorbent core 24, as shown in FIG. 30. As there shown, the respective outermost edges of each of ends 126, 128 of the napkin preferably are not in overlapped condition, but are either in contacting relationship, or they can be slightly spaced from each other, as visible in the right side view of the folded napkin as it is illustrated in FIG. 31. After both ends 126, 128 have been folded inwardly, each of flaps 136, 138 is individually folded inwardly about suitable respective fold lines that extend generally parallel to longitudinal axis 38, so that the adjacent first and second fastener elements 132, 134 contact each other to provide a completely closed, folded assembly in the form shown in FIG. 32, in which flaps 136 and 138 are not overlapped, to minimize the overall thickness of the completely-folded napkin. Although illustrated and described in the context of pairs of fastener elements 132, 134 on respective ends and flaps of the napkins, unitary fastener elements (not shown) can be substituted for each pair of fastener elements shown, if desired.

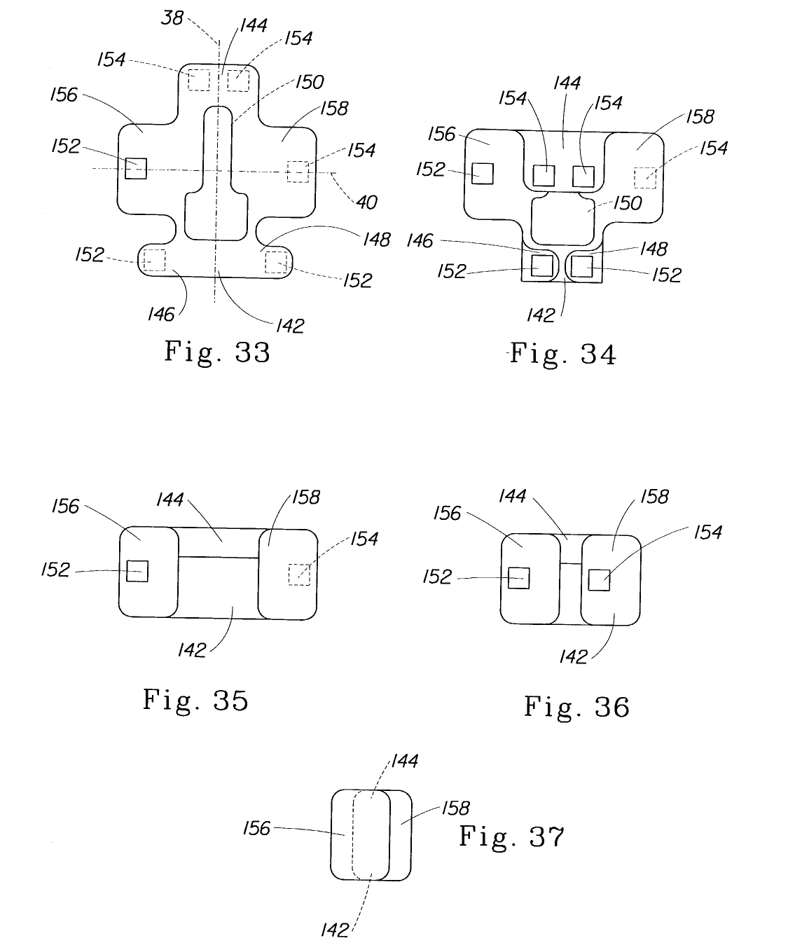

A further embodiment of the present invention is shown in FIGS. 33 and 34, which illustrate an asymmetrical napkin 140 having a greater width at end 142 than at end 144. Wider end 142 includes outwardly extending lobes 146, 148 to accommodate a larger absorbency area and absorbency volume by permitting an asymmetric absorbent core 150 having an enlarged end to be employed. Absorbent core 150 is preferably asymmetrical relative to transverse axis 40 but is preferably symmetrical relative to longitudinal axis 38. Each of lobes 146, 148 carries on the garment-facing surface of the lobe one fastener element 152. Similarly, the narrower, opposite end 144 of napkin 140 includes on the garment-facing surface of that end a pair of laterally spaced, complementary fastener elements 154 that are complementary with and engageable with fastener elements 152 carried on the backsheet side of each of lobes 146, 148. In that regard, if desired a single, elongated fastener element (not shown), having a longitudinal axis that extends transversely relative to the longitudinal axis of core 150, can be provided instead of the two spaced fastener elements 154.

Lobes 146, 148, ends 142, 144, and flaps 156, 158 of the FIG. 33 embodiment can be folded inwardly to provide a closed, folded napkin in a manner similar to the folding sequence generally illustrated in FIGS. 25 through 28. However, initially lobes 146 and 148 are each sequentially folded inwardly about respective fold lines that extend in a generally parallel direction relative to longitudinal axis 38, so that each of the lobes overlies absorbent core 150 with its respective fastener element 152 facing upwardly, as shown in FIG. 34. After lobes 146, 148 have been folded over, they are not in overlapped relationship, and their outermost edges can be spaced from each other, as shown in FIG. 34, although the outermost edges of the folded-over lobes can also be in contact with each other, if desired.

After lobes 146, 148 have been folded inwardly, narrow end 144 is then folded inwardly about a generally transversely-extending fold line to overlie absorbent core 150. After narrow end 144 has been folded over, fastener elements 154 carried by narrow end 144 face upwardly, as shown in FIG. 34. Additionally, it is preferred that narrow end 144 be so oriented after folding that fastener elements 154 carried by narrow end 144 can engage fastener elements 152 carried by lobes 146, 148.

Wide end 142 that includes lobes 146, 148 is then folded inwardly about a transversely extending fold line to overlie absorbent core 150. The fold line is preferably so selected that when wide end 142 is folded over, fastener elements 152 carried by lobes 146, 148 overlie the respective complementary fastener elements 154 on the upwardly-facing surface of folded-over narrow end 144. When folded-over wider end 142 is pressed against folded-over narrow end 144, the respective fastener elements 152, 154 engage each other to hold and to maintain the narrow and wide ends of napkin 140 in partially overlapped relationship, as shown in FIG. 35. Thereafter, flap 158 is folded inwardly over folded-over end 142, as shown in FIG. 36, to expose fastener element 154 carried by flap 158, and flap 156 is then folded inwardly over flap 158, as shown in FIG. 37, so that fastener element 152 carried by flap 156 engages with fastener element 154 carried by flap 158 to securely hold the folded napkin in its folded condition.

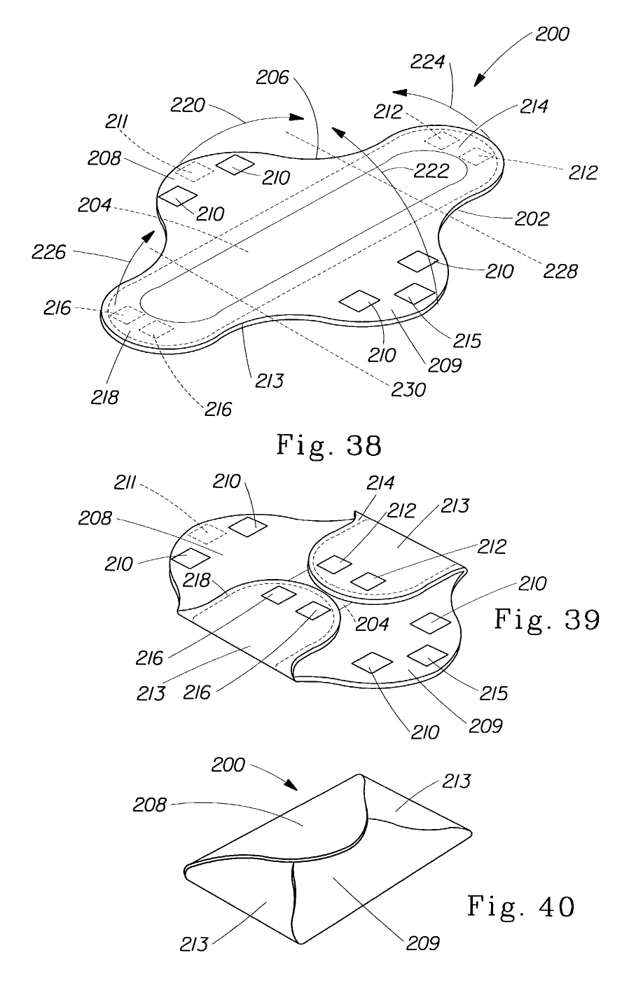

A still further embodiment of the present invention that includes flaps, that is foldable into a compact, self-contained package, and that does not require fastener cover strips or release strips is shown in FIG. 38. Sanitary napkin 200 is structurally similar to the napkin shown in FIGS. 1 and 2 in that it includes a body-facing surface, a garment-facing surface, a fluid-impervious backsheet 202 defining the garment-facing surface, an elongated absorbent core 204, a fluid-pervious topsheet 206 defining the body-facing surface, and a pair of laterally outwardly extending flaps 208 and 209. Absorbent core 204 can be of a thinner overall structure, comparable with commercially-available napkins commonly referred to as “mini-pads.”

Each of flaps 208 and 209 includes a pair of first fastener elements 210 positioned on the body-facing surface of napkin 200 in longitudinally spaced relationship. Flaps 208 and 209 also include a second fastener element 211 that is positioned between first fastener elements 210. On flap 208 fastener element 211 is on the garment-facing side of the flap and on flap 209 fastener element 215 is on the body-facing side of the flap. Preferably, fastener elements 210 and 211 are areas of pressure-sensitive adhesive.

The FIG. 38 embodiment also includes a third pair of fastener elements 212 positioned in laterally spaced relationship on the garment-facing surface of release strip 213 adjacent first longitudinal end 214 of napkin 200, and a fourth pair of fastener elements 216 positioned in laterally spaced relationship on the garment-facing surface of the backsheet adjacent second longitudinal end 218 of napkin 200. Fastener elements 212 and 216 are also preferably pressure-sensitive adhesive areas. Instead of two individual, laterally spaced adhesive areas as shown, a single, elongated adhesive area having its longitudinal axis extending transversely relative to the napkin longitudinal axis can be provided as an alternative arrangement, if desired.

The manipulative steps to fold napkin 200 into a compact, self-contained package are similar to those illustrated in FIGS. 25 through 28. Each of ends 214 and 218 is first individually folded inwardly in the directions indicated by arrows 224 and 226, respectively, about respective imaginary fold lines 228 and 230, so that ends 214 and 218 are as shown in FIG. 39, in which each end overlies absorbent core 204, but the ends are not necessarily in overlapped relationship relative to each other. When ends 214 and 218 are folded over in the manner described, third fastener elements 212 and fourth fastener elements 216 are exposed and are facing upwardly, in the same direction as the body-facing surface of absorbent core 204, as shown in FIG. 39.

Flap 208 is then folded inwardly in the direction shown by arrow 220, so that it overlies a portion of folded-over ends 214 and 218 with fastener element 211 facing upwardly. Similarly, flap 209 is then folded inwardly in the direction shown by arrow 222, so that it overlies a portion of folded-over ends 214 and 218 with fastener element 215 facing upwardly, as shown in FIG. 40. When so folded over, first fastener elements 210 of each of flaps 208 and 209 engage with and releasably adhere to respective third and fourth fastener elements 212 and 216, and fastener elements 211 and 215 engage with each other to retain the folded-over elements in their folded-over positions.

The resulting folded-over napkin is of a convenient size, in which each of the fastener elements is contained within the folded-over napkin and no fastener elements are exposed. Additionally, the body-facing surface of the absorbent core is completely contained within the folded-over napkin to maintain the cleanliness of the absorbent core before use of the napkin and to substantially cover the absorbent core after use of the napkin.

It will be apparent that the napkin embodiments illustrated in FIGS. 25, 29, 33, and 38 do not require release liner sheets to cover the flap and end adhesive areas, because when in their completely folded condition each of those embodiments is without outwardly-facing adhesive areas.

### Flap Severance Embodiment

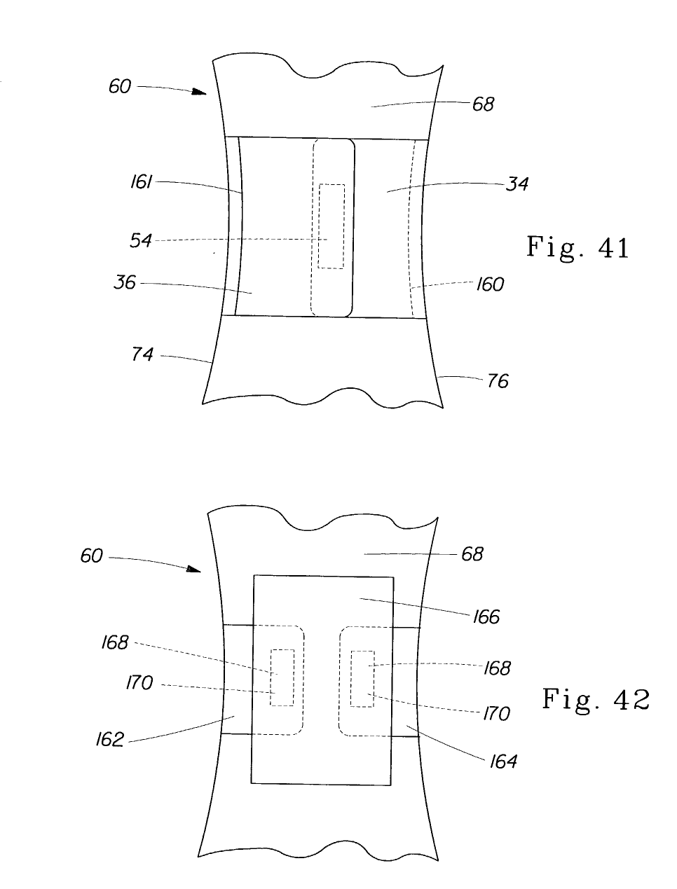

In a still further embodiment of the present invention, as illustrated in FIG. 41, at least one of flaps 34, 36 includes a line of weakness 160 that extends across the flap, and that can be oriented to extend in a direction generally parallel with the longitudinal axis of the napkin. The napkin can have an overall structure as shown in FIGS. 1 and 2, and line of weakness 160 can be straight, curved, or it can include both straight and curved components.

FIG. 41 is a fragmentary view of the outermost surface of the crotch area of a panty 60 with a napkin (not shown) in position against the innermost surface at the crotch area. Flaps 34 and 36 have each been folded over respective edges 76, 74 of the leg openings at the crotch area of the panty and they have been attached to each other by one or more fastener elements 54, only one of which is shown in FIG. 39. Line of weakness 160 is provided to facilitate removal of the napkin after use, and without the need by the wearer to disconnect the flaps from each other at fastener element 54. Removal of the napkin is effected by manually tearing the flap that includes line of weakness 160, which in this instance is flap 34. The tear is made along line of weakness 160 to sever flap 34 to release the flap interconnection that assists in holding the napkin in position relative to the crotch area of the panty, and thereby to facilitate removal of the napkin from the panty without the need to disconnect the flaps from each other at fastener element 54. Line of weakness 160 is preferably a line that is formed in the material that defines flap 34, or flap 36, or both, and it enables the user to easily sever the flap in which line of weakness 160 is provided.

Line of weakness 160 can be a score line in the form of a narrow line of reduced flap material thickness that can be impressed into the flap material by heat, by pressure, or by a combination of heat and pressure, so that the flap material can be readily torn along that line without excessive effort. Preferably, line of weakness 160 retains sufficient structural integrity to maintain as a single piece the flap in which the line of weakness is formed, until severance of that line is desired. As an alternative structural embodiment, line of weakness 160 can be a series of spaced, aligned embossments of any suitable shape that together define a series of spaced, thinner, weakened areas in the flap material. As a further alternative, line of weakness 160 can be defined by a series of aligned, spaced, linear cuts that extend through the flap material, or it can be provided by a series of aligned, non-linear cuts or small apertures, such as perforations, that extend through the flap material and that can be of any desired and effective form.

As shown in FIG. 41 line of weakness 160 provided in flap 34 is a series of aligned, spaced, linear cuts that extend through the flap material, and line of weakness 161 provided in flap 36 is a score line defined by a line of reduced flap material thickness. Additionally, although shown in FIG. 39 as having two differently structured lines of weakness, merely for illustrative purposes, it should be understood that if two lines of weakness are provided, they preferably are of the same structure. In its preferred form the napkin need have only a single line of weakness to provide the desired ease of removability of the napkin after use.

Preferably, the line of weakness is a score line of reduced flap material thickness in order to maintain the liquid-impervious nature of the garment-contacting surface of the flap in which the line of weakness is provided. In any event, however, the line of weakness is so formed that a wearer can readily and without excessive effort tear the flap to sever the encircling assembly of flaps 34 and 36 along the line of weakness for more convenient and more rapid removal of a used napkin.

### Flap Connector Piece Embodiment

Another embodiment of the present invention is illustrated in FIG. 42, which is another fragmentary view of the outermost surface of the crotch area of a panty 60 with a napkin (not shown) in position against the innermost surface at the crotch area. In the embodiment shown in FIG. 42, napkin flaps 162, 164 do not meet when folded over crotch area edges 74, 76 of panty 60, but, instead, they are interconnected by a separate connector piece 166. Each flap can carry on its body-contacting surface, adjacent the outermost end of the flap, one element 168 of a fastener system. Connector piece 166 can include a pair of complementary fastener elements 170 that are adapted and positioned to contact and to engage with complementary fastener elements 168 carried by the flaps. The complementary fastener elements can be of the hook-and-loop type, if desired. Alternatively, either the connector piece or the flaps, or both, can be provided with adhesive areas to permit interconnection between the flaps and the connector piece.

Connector piece 166 can be of generally rectangular form, as shown, or it can be of any other convenient and effective form, such as square, circular, oval, or the like, and it can be made from any soft, flexible material having sufficient strength to be self-supporting. Such a connector piece can be made to be washable and reusable, and it can incorporate an enhanced fastening system for additional attachment security.

The materials from which connector piece 166 can be made include the backsheet material, as well as a combination of the backsheet material together with the topsheet material. In that regard, trip strips of backsheet or of backsheet and attached top sheet material remaining after cutting napkin components from webs of backsheet material or overlapped backsheet and topsheet material, when cut to appropriate size, can be used as the connector piece. Preferably, the connector piece is formed from a material that can readily be disposed of without environmental detriment.

The connector piece can also be made from a durable material that can be reused. In that instance a single connector piece can be packaged along with a number of sanitary napkins to reduce cost.

Although particular embodiments of the present invention have been illustrated and described, it will be apparent to those skilled in the art that various changes and modifications can be made without departing from the spirit of the present invention. It is therefore intended to encompass within the appended claims all such changes and modifications that fall within the scope of the present invention.

What is claimed is:

1. A disposable absorbent article adapted to be worn and to be positioned relative to the wearer's body for receiving and retaining body waste fluids to prevent soiling of undergarments worn by the wearer, said absorbent article comprising:
a. a liquid-impervious backsheet having a rear surface adapted to face toward the wearer's garments when the absorbent article is worn, and a front surface adapted to face toward the wearer's body when the absorbent article is worn, the backsheet including a pair of longitudinally extending side edges;
b. an elongated absorbent core for absorbing body waste fluids of the wearer while the absorbent article is worn, the absorbent core positioned in overlying relationship with the front surface of the backsheet and having a longitudinal axis and a transverse axis;
c. a liquid-pervious topsheet overlying the absorbent core and having a rear surface facing toward the absorbent core and a front surface facing toward and adapted to contact the wearer's body when the article is worn;
d. a pair of side flaps extending laterally outwardly from the side edges of the absorbent core, each flap having a liquid-impervious outer surface adapted to face toward the wearer's garments when the absorbent article is worn, and an inner surface adapted to face toward the wearer's body when the absorbent article is worn, wherein each of the flaps extends laterally from the absorbent core longitudinal axis a distance sufficient to enable the flaps to be in at least partial overlapping relationship when each of the flaps is folded around respective edges of a crotch area of the undergarment after initial placement of the backsheet of the absorbent article in a desired position against the inner crotch surface of the undergarment, at least one side flap having a first area of elastic extensibility defined by a plurality of laterally spaced, longitudinally extending, first parallel ridges and alternating parallel grooves formed in the at least one side flap to allow the at least one side flap to elastically extend in response to stresses applied to the at least one side flap when the absorbent article is placed in position against the inner crotch surface of the undergarment.

2. A disposable absorbent article in accordance with claim 1 wherein the parallel ridges extend substantially parallel to the absorbent core longitudinal axis.

3. A disposable absorbent article in accordance with claim 2 wherein the ridges lie outwardly of and adjacent to lateral side edges of the absorbent core.

4. A disposable absorbent article in accordance with claim 3 wherein the extensibility areas are provided in each side flap.

5. A disposable absorbent article in accordance with claim 1 including second extensibility areas having second groups of a plurality of alternating ridges and grooves formed in the at least one side flap, wherein the ridges and grooves defining the second extensibility areas are positioned adjacent longitudinal ends of the first extensibility areas.

6. A disposable absorbent article in accordance with claim 5 wherein the ridges and grooves defining the second extensibility areas are provided in each side flap.

7. A disposable absorbent article in accordance with claim 6 wherein the ridges and grooves defining the second extensibility areas have centerlines that extend substantially perpendicular to the absorbent core longitudinal axis.

8. A disposable absorbent article in accordance with claim 7 wherein the ridges and grooves defining the second extensibility areas define a triangular area in each side flap.

9. A disposable absorbent article in accordance with claim 8 wherein the ridges and grooves defining the second extensibility areas increase in length in a direction from outer ends of the core inwardly toward the absorbent core transverse axis.

10. A disposable absorbent article in accordance with claim 5 wherein the ridges and grooves defining the second extensibility areas have centerlines that are inclined at an acute angle relative to the absorbent core longitudinal axis.

11. A disposable absorbent article in accordance with claim 10 wherein the acute angle is about 45°.

12. A disposable absorbent article in accordance with claim 10 wherein the ridges and grooves defining the second extensibility areas are of substantially equal length.

13. A disposable absorbent article in accordance with claim 10 wherein the ridges and grooves defining the second extensibility areas have centerlines that extend substantially perpendicular to a line passing through the center of the article and bisecting the longitudinal and transverse axes.

14. A disposable absorbent article in accordance with claim 13 wherein the ridges and grooves defining the second extensibility areas are of unequal length.

15. A disposable absorbent article in accordance with claim 14 wherein the ridges and grooves defining the second extensibility areas define a triangular area of elastic extensibility.

16. A disposable absorbent article in accordance with claim 15 wherein the ridges and grooves defining the second extensibility areas decrease in length in a direction from adjacent a flap edge toward the absorbent core.

17. A disposable absorbent article in accordance with claim 16 wherein the ridges and grooves defining the second extensibility areas define an isosceles triangle.

18. A disposable absorbent article in accordance with claim 1 wherein the side flaps are integrally formed with the backsheet.

19. A disposable absorbent article in accordance with claim 1 wherein the side flaps are defined by a flexible, unitary crosspiece in sheet form that is secured to the front surface of the backsheet and that extends laterally outwardly therefrom.

20. A disposable absorbent article in accordance with claim 1 wherein the side flaps are defined by a pair of discrete sheets of flexible material that are each secured to the front surface of the backsheet and that each extend laterally outwardly therefrom.

21. A disposable absorbent article adapted to be worn and to be positioned relative to the wearer's body for receiving and retaining body waste fluids to prevent soiling of undergarments worn by the wearer, said absorbent article comprising:
a. a liquid-impervious backsheet having a rear surface adapted to face toward the wearer's garments when the absorbent article is worn, and a front surface adapted to face toward the wearer's body when the absorbent article is worn, the backsheet including a pair of longitudinally extending side edges;
b. an elongated absorbent core for absorbing body waste fluids of the wearer while the absorbent article is worn, the absorbent core positioned in overlying relationship with the front surface of the backsheet and having a longitudinal axis and a transverse axis;
c. a liquid-pervious topsheet overlying the absorbent core and having a rear surface facing toward the absorbent core and a front surface facing toward and adapted to contact the wearer's body when the article is worn;
d. a pair of side flaps extending laterally outwardly from the side edges of the absorbent core, each flap having a liquid-impervious outer surface adapted to face toward the wearer's garments when the absorbent article is worn, and an inner surface adapted to face toward the wearer's body when the absorbent article is worn, wherein each of the flaps has a lateral extent from the longitudinal axis sufficient to enable the flaps to be in at least partial overlapping relationship when each of the flaps is folded around respective edges of a crotch area of the undergarment after initial placement of the backsheet of the absorbent article in a desired position against the inner crotch surface of the undergarment;
e. a fastener element positioned on the flap outer surface of at least one flap to enable contact to be made between the fastener element of the at least one flap and the other flap after the flaps have each been folded around the crotch area of the undergarment, wherein at least one side flap includes a line of weakness extending across the flap in a direction generally parallel to the absorbent core longitudinal axis to permit severance of the at least one flap along the line of weakness to facilitate separation of the contacting flaps and enable removal of the absorbent article from the undergarment.

22. A disposable absorbent article in accordance with claim 21 wherein the line of weakness is defined by a series of aligned, spaced embossments formed in the at least one side flap, the embossments defining areas of reduced flap thickness.

23. A disposable absorbent article in accordance with claim 21 wherein the line of weakness is a score line formed in the at least one side flap, wherein the score line is defined by a line of reduced flap material thickness.

24. A disposable absorbent article in accordance with claim 21 wherein the line of weakness is defined by a series of spaced perforations.

25. A disposable absorbent article in accordance with claim 21 wherein the spaced perforations are aligned linear cuts that extend through the at least one flap.

26. A disposable absorbent article in accordance with claim 21 wherein a line of weakness is formed in each side flap.

27. A disposable absorbent article in accordance with claim 26 wherein the lines of weakness are score lines defined by lines of reduced flap thickness.

28. A disposable absorbent article in accordance with claim 26 wherein the lines of weakness are aligned, spaced embossments in the flap, the embossments defining areas of reduced flap thickness.

29. A disposable absorbent article in accordance with claim 26 wherein the lines of weakness are aligned, spaced perforations extending through the flap material.

30. A disposable absorbent article in accordance with claim 29 wherein the spaced perforations are linear cuts.

31. A disposable absorbent article adapted to be worn and to be positioned relative to the wearer's body for receiving and retaining body waste fluids to prevent soiling of a garment worn by the wearer of the absorbent article, said absorbent article comprising:
a. a body portion having an absorbent core adapted to be positioned adjacent the body of the wearer for absorbing body fluids, and a liquid-impervious backsheet overlying the absorbent core and having a garment-facing side, the backsheet adapted to be positioned between the absorbent core and a garment worn by the wearer of the article with the garment-facing side facing away from the wearer's body;
b. a pair of side flaps extending laterally outwardly from the body portion of the article and having a lateral length sufficient to permit the flaps to extend over an outer surface of the crotch area of the garment and to cover crotch edge portions at the crotch area to protect the crotch edge portions from being soiled by body fluids that have not been retained in the absorbent core, the side flaps each carrying a first fastener element adjacent laterally outermost edges of the flap on the garment-facing side of the article; and
c. a connector member having a length sufficient to extend between and to contact outwardly-facing surfaces of the side flaps after the flaps have been folded over the crotch area edges of the garment, the connector member including a pair of spaced, second fastener elements that are releasably engageable with the first fastener elements to hold the flaps in a folded-over condition relative to the crotch area of the garment and to maintain the article in a desired position relative to the garment.

32. A disposable absorbent article adapted to be worn and to be positioned relative to the wearer's body for receiving and retaining body waste fluids to prevent soiling of undergarments worn by the wearer, said absorbent article comprising:
a. a liquid-impervious backsheet having a rear surface adapted to face toward the wearer's garments when the absorbent article is worn, and a front surface adapted to face toward the wearer's body when the absorbent article is worn, the backsheet including a pair of longitudinally extending side edges;
b. an elongated absorbent core for absorbing body waste fluids of the wearer while the absorbent article is worn, the absorbent core positioned in overlying relationship with the front surface of the backsheet and having a longitudinal axis and a transverse axis;
c. a liquid-pervious topsheet overlying the absorbent core and having a rear surface facing toward the absorbent core and a front surface facing toward and adapted to contact the wearer's body when the article is worn, wherein the absorbent core is asymmetric about its transverse axis and includes at a first longitudinal end a pair of lobes of absorbent material that extend laterally outwardly relative to a core longitudinal axis a greater distance than a corresponding width of the core at a second, opposite longitudinal end of the core to provide additional absorptive capacity at the first longitudinal end of the core, wherein each lobe includes an outer, garment-facing surface that faces the wearer's undergarment when the article is worn and a first core fastener element carried on the garment-facing surfaces of each of the lobes, and wherein the second longitudinal end of the core includes an outer, garment-facing surface that faces the wearer's undergarment when the article is worn, and at least one second core fastener element carried on the garment-facing surface of the core adjacent the second longitudinal end thereof, wherein the at least one second core fastener element is releasably engageable with each of the first core fastener elements for releasably retaining the core in a folded-over condition before and after use of the article.

33. A disposable absorbent article adapted to be worn and to be positioned relative to the wearer's body for receiving and retaining body waste fluids to prevent soiling of garments worn by the wearer, said absorbent article comprising:
a. a liquid-impervious backsheet having a rear surface adapted to face toward the wearer's garments when the absorbent article is worn, and a front surface adapted to face toward the wearer's body when the absorbent article is worn, the backsheet including a pair of longitudinally extending side edges;
b. an elongated absorbent core for absorbing body waste fluids of the wearer while the absorbent article is worn, the absorbent core positioned in overlying relationship with the front surface of the backsheet and having a longitudinal axis and a transverse axis;
c. a liquid-pervious topsheet overlying the absorbent core and having a rear surface facing toward the absorbent core and a front surface facing toward and adapted to contact the wearer's body when the article is worn;
d. a pair of side flaps extending laterally outwardly from the side edges of the absorbent core, each flap having a liquid-impervious outer surface adapted to face toward the wearer's garments when the absorbent article is worn, and an inner surface adapted to face toward the wearer's body when the absorbent article is worn, wherein each of the flaps has a lateral extent from the longitudinal axis sufficient to enable the flaps to be in at least partial overlapping relationship when each of the flaps is folded around respective edges of a crotch area of the undergarment after initial placement of the backsheet of the absorbent article in a desired position against the inner crotch surface of the undergarment;
e. at least one first fastener element positioned on the inner surface of each flap, and at least one second fastener element complementary with the at least one first fastener element and positioned on the outer surface of the article adjacent each longitudinal end thereof, wherein the first and second fastener elements are attachable to each other, releasable from each other, and subsequently reattachable to each other, whereby the ends and flaps of the article can be folded over the absorbent core to protect the core before use and can be folded over the core after use for sanitary disposal of a used article.

34. A disposable absorbent article in accordance with claim 33 wherein the at least one first fastener element is a pair of spaced first fastener elements, and wherein the at least one second fastener element is a pair of spaced second fastener elements.

35. A method of folding a disposable absorbent article for convenient carrying before and after use, the article including an elongated absorptive core having a body-facing surface and a garment-facing surface, and a first, narrow end and a second, wide end defined by a pair of laterally outwardly extending lobes that include absorptive material, the article having a pair of laterally outwardly extending first and second side flaps positioned between the first and second ends of the core and spaced from the lobes along a longitudinal axis of the core, said method comprising the steps of:
a. providing a first core fastener element on the garment-facing surface of each lobe;
b. providing at least one second core fastener element on the garment-facing surface at the first end of the core, wherein the first and second core fastener elements are releasably engageable with each other;
c. providing a first flap fastener element on the body-facing surface of the first flap and a second flap fastener element on the garment-facing surface of the second flap, wherein the first and second flap fastener elements are releasably engageable with each other;
d. folding each of the lobes at the second end of the core inwardly toward the core longitudinal axis to overlie the core;
e. folding the first end of the core toward the folded-over second end of the core so that the at least one second core fastener element is in opposed relationship with the first core fastener element;
f. pressing the opposed first and second core fastener elements together to cause them to engage each other and to retain the core in folded-over condition so that the body-facing surface of the elongated absorptive core is contained within the folded-over core;
g. folding the second flap inwardly to overlie the folded-over core so that the second flap fastener element faces outwardly of the folded-over core;
h. folding the first flap inwardly to overlie the folded-over second flap so that the first and second flap fastener elements are in opposed relationship; and
i. pressing the opposed first and second flap fastener elements together to cause them to engage each other and to retain the flaps in folded-over condition to provide an enclosed absorbent article for convenient and sanitary handling before and after use.

36. A method of folding a disposable absorbent article for convenient carrying before and after use, the article including an elongated body having a substantially centrally positioned absorptive core, the elongated body having a body-facing surface and a garment-facing surface and a first end and a second end, a pair of laterally outwardly extending first and second side flaps positioned between the first and second ends of the elongated body, a first body fastener element on the garment-facing surface at each end of the elongated body, and a first flap fastener element on the body-facing surface of the first flap and a second flap fastener element on the garment-facing surface of the second flap, wherein the first and second flap fastener elements are releasably engageable with each other, said method comprising the steps of:
a. folding the first end of the elongated body inwardly toward an article transverse axis and about an imaginary fold line positioned between the article transverse axis and the first end of the elongated body to overlie a portion of the elongated body so that the first core fastener element faces in an upward direction relative to the elongated body;
b. folding the second end of the core inwardly toward the article transverse axis and about an imaginary fold line positioned between the article transverse axis and the second end of the elongated body to overlie a portion of the elongated body so that the second body fastener element faces in an upward direction relative to the elongated body;
c. folding the first flap inwardly to overlie the core so that the first fastener element faces outwardly relative to the folded-over core;
d. folding the second flap inwardly to overlie the folded-over first flap so that the first flap fastener element contacts and engages with the second flap to retain the first and second flaps in folded-over condition, whereby the body-facing surface of the elongated absorptive core is contained within the folded-over core to provide an enclosed absorbent article for convenient and sanitary handling before and after use.
